(19) United States
(12) Patent Application Publication
(10) Pub. No.: US 2011/0270345 A1
(43) Pub. Date: Nov. 3, 2011

(54) FOCAL NONINVASIVE STIMULATION OF THE SENSORY CORTEX OF A SUBJECT WITH CEREBRAL PALSY

(75) Inventors: Michael VanDoren Johnston, Baltimore, MD (US); Alexander H. Hoon, JR., Ellicott City, MD (US)

(73) Assignee: Johns Hopkins University, Baltimore, MD (US)

(21) Appl. No.: 12/673,006

(22) PCT Filed: Aug. 11, 2008
(86) PCT No.: PCT/US08/09581

§ 371 Date: Mar. 29, 2010

Related U.S. Application Data
(60) Provisional application No. 60/964,259, filed on Aug. 11, 2007.

(51) Int. Cl. A61N 1/05

(52) U.S. Cl. 607/45

(57) ABSTRACT

Disclosed are methods and related devices for use with subjects with cerebral palsy or periventricular leukomalacia. In preferred embodiments, diffusion tensor imaging (DTI) is used to identify neural areas and transcranial magnetic stimulation (TMS) is used to stimulate neural pathways.

### CROSS-REFERENCE TO RELATED APPLICATIONS

This application claim priority to U.S. Provisional Application Ser. No. 60/964,259 filed 11 Aug. 2007, which is fully incorporated by reference herein.

### STATEMENT OF GOVERNMENTAL SUPPORT

This invention was supported by Federal funding, thus the US Government has certain rights herein. This work was supported by the National Institutes of Health (NIH) grant RO1 AG20012, P41 R15241, the National Center for Research Resources (NCRR), Grant #M01-RR00052.

### FIELD OF THE INVENTION

This invention relates to the nervous system, more particularly to the stimulation of neurological cells in subjects with cerebral palsy.

### BACKGROUND OF THE INVENTION

The term cerebral palsy (CP) describes motor impairment attributable to early injury to the developing brain, encompassing pre-, peri-, and postnatal etiologies. (Osler S W. The Cerebral Palsies of Children. London, UK: Mac Keith Press; 1987; Keogh J M, Badawi N. The origins of cerebral palsy. Curr Opin Neurol 2006; 19:129-34)

CP is the second-most expensive developmental disability to manage over the course of a person's lifetime (second to mental disabilities), with an average lifetime cost per person of USD$921,000 (in 2003 dollars). The incidence in the six countries surveyed is approximately an average of 2.12-2.45 per 1000 live births; there has been a slight increase in recent years due to an increase in premature births, in which the incidence is as high as 6/1000 births. Although improvements in neonatal nursing help reduce the number of babies who develop cerebral palsy, they also mean that babies with very low birth weights survive, and these babies are more likely to have cerebral palsy.

Babies born with severe CP often have an irregular posture; their bodies may be either very floppy or very stiff. Birth defects, such as spinal curvature, a small jawbone, or a small head sometimes occur along with CP. Symptoms may appear, change, or become more severe as a child gets older. Some babies born with CP do not show obvious signs right away. The effects of cerebral palsy fall on a continuum of motor dysfunction that may range from virtually unnoticeable to “clumsy” and awkward movements on one end of the spectrum to such severe impairments that coordinated movements are almost impossible on the other end of the spectrum. Secondary conditions can include seizures, epilepsy, speech or communication disorders, eating problems, sensory impairments, mental retardation, learning disabilities, and/or behavioral disorders.

In order for bones to attain their normal shape and size, they require the stresses from normal musculature. Osseous findings will therefore mirror the specific muscular deficits in a given person with CP. The shafts of the bones are often thin (gracile). When compared to these thin shafts (diaphyses) the metaphyses often appear quite enlarged (ballooning). With lack of use, articular cartilage may atrophy, leading to narrowed joint spaces. Depending on the degree of spasticity, a person with CP may exhibit a variety of angular joint deformities. Because vertebral bodies need vertical gravitational loading forces to develop properly, spasticity and an abnormal gait can hinder proper and/or full bone and skeletal development. People with CP tend to be shorter than the average person because their bones are not allowed to grow to their full potential. Sometimes bones grow at different lengths, so the person may have one leg longer than the other.

Onset of arthritis and osteoporosis can occur much sooner in adults with CP. CP's resultant motor disorder(s) are sometimes, though not always, accompanied by “disturbances of sensation, cognition, communication, perception, and/or behavior, and/or by a seizure disorder”. (“United Cerebral Palsy Research and Educational Foundation”. Retrieved on 2007 Jul. 29; Bax M, Goldstein M, Rosenbaum P, et al (2005). “Proposed definition and classification of cerebral palsy, April 2005”. Developmental medicine and child neurology 47 (8): 571-6. doi:10.1017/S001216220500112X. PMID 16108461)

Cerebral palsy (CP) is an umbrella term encompassing a group of non-progressive, non-contagious conditions that cause physical disability in human development. All types of CP are characterized by abnormal muscle tone, posture (i.e. slouching over while sitting), reflexes, or motor development and coordination. There can be joint and bone deformities and contractures (permanently fixed, tight muscles and joints). The classical symptoms are spasticity, spasms, other involuntary movements (e.g. facial gestures), unsteady gait, problems with balance, and/or soft tissue findings consisting largely of decreased muscle mass. Overall, CP symptomatology is very diverse.

Etiology

Heretofore, CP is believed to be caused by damage to the motor control centers of the young developing brain and can occur during pregnancy (about 75 percent), during childbirth (about 5 percent) or after birth (about 15 percent) up to about age three.

It is a non-progressive disorder, meaning the brain damage does not worsen, but secondary orthopedic difficulties are common. There is no known cure for CP. Medical intervention is limited to the treatment and prevention of complications arising from CP's effects.

Periventricular leukomalacia (PVL) refers to the most common CP-related brain injury in premature neonates. PVL is related to the susceptibility of the periventricular white matter to focal ischemic and/or infectious/inflammatory destructive processes occurring between 24 and 34 weeks of gestation. (Volpe J J. Cerebral white matter injury of the premature infant: more common than you think. Pediatrics 2003; 112:176-80)

A more diffuse noncystic injury to immature oligodendrocytes is now increasingly recognized in infants discharged from modern neonatal intensive care units. Associated abnormalities may include reductions in cortical gray matter, deep gray matter and posterior fossa injury. (Volpe J J. Cerebral white matter injury of the premature infant: more common that you think. Pediatrics 2003; 112:176-80; Miller S P, Cozzio C C, Goldstein R B, et al. Comparing the diagnosis of white matter injury in premature newborns with serial MR imaging and transfontanel ultrasonography findings. AJNR Am J Neuroradiol 2003; 24:1661-69; Folkerth R D. Neuropathologic substrate of cerebral palsy. J Child Neurol 2005; 20:940-49; Johnsen S D, Bodesnteinser J B, Lotze T E. Frequency and nature of cerebellar injury in the extremely premature survivor with cerebral palsy. J Child Neurol 2005; 20:60-64; Srinivasan L, Dutta R, Counsell S J, et al. Quantification of deep gray matter in preterm infants at term-equivalent age using manual volume try of 3-tesla magnetic resonance images. Pediatrics 2007; 119:759-65)

Neuropathologic data reveal coagulative necrosis in the periventricular white matter with diffuse glial injury or focal injuries that can potentially cavitate. (Inder T E, Volpe J J. Mechanisms of perinatal brain injury. Semin Neonatol 2000; 5:3-16; Rezaie P, Dean A. Periventricular leukomalacia, inflammation and white matter lesions within the developing nervous system. Neuropathology 2002; 22: 106-32; Folkerth R D, Keefe R J, Haynes R L, et al. Interferon-gamma expression in periventricular leukomalacia in the human brain. Brain Pathol 2004; 14: 265-74)

CP Classification

CP is divided into four major classifications to describe the different movement impairments. These classifications reflect the area of brain damaged. The four major classifications are: Spastic, Athetoid/Dyskinetic, Ataxic, and Mixed. In 30 percent of all cases of CP, the spastic form is found along with one of the other types. There are a number of other, less prevalent types of CP, but these are the most common.

Spastic CP:

Spastic (ICD-10 G80.0-G80.1) cerebral palsy is by far the most common type, occurring in 70% to 80% of all cases. People with this type are hypertonic and have a neuromuscular condition believed to stem from damage to the corticospinal tract or the motor cortex that affects the nervous system's ability to receive gamma amino butyric acid in the area(s) affected by the spasticity. Occasionally, terms such as monoplegia, paraplegia, triplegia, and pentaplegia may also be used to refer to specific manifestations of the spasticity. However, spastic CP is generally classified by topography dependent on the region of the body affected; these include: With spastic hemiplegia one side is affected. Generally, injury to the left side of the brain will cause a right sided deficit, and vice versa.

In spastic diplegia the lower extremities are generally affected more than the upper extremities. Most people with spastic diplegia do eventually walk. The gait of a person with spastic diplegia is typically characterized by a crouched gait. Toe walking and flexed knees are common. Hip problems, dislocations, and side effects like strabismus (crossed eyes) are common. Strabismus affects three quarters of people with spastic diplegia. This is due to weakness of the muscles that control eye movement. In addition, these individuals are often nearsighted. In many cases the IQ of a person with spastic diplegia is unaffected by the condition.

With spastic quadriplegia the whole body is affected, and all four limbs affected equally. Some children with quadriplegia also have hemiparetic tremors; an uncontrollable shaking that affects the limbs on one side of the body and impairs normal movement. Autonomic dysreflexia can be caused by hardened feces, urinary infections, and other problems, resulting in the overreaction of the nervous system and can result in high blood pressure. Blockage of tubes inserted into the body to drain or enter fluids also needs to be monitored to prevent autonomic dysreflexia in quadriplegia. The proper functioning of the digestive system needs to be monitored as well.

Ataxic CP:

Ataxia (ICD-10 G80.4) type symptoms can be caused by damage to the cerebellum. Forms of ataxia are less common types of Cerebral Palsy, occurring in at most 10% of all cases. Some of these individuals have hypotonia and tremors. Motor skills like writing, typing, or using scissors might be difficult, as well as problems with balance, especially while walking. It is common for individuals to have difficulty with visual and/or auditory processing of objects.

Athetoid/Dykinetic CP:

Athetoid or dyskinetic (ICD-10 G80.3) is mixed muscle tone—sometimes hypertonia and sometimes hypotonia. Hypotonia will usually occur before 1 year old; the muscle tone will be increased with age and progress to Hypertonia. People with athetoid CP have trouble holding themselves in an upright, steady position for sitting or walking, and often show involuntary motions. For some people with athetoid CP, it takes a lot of work and concentration to get their hand to a certain spot (like scratching their nose or reaching for a cup). Because of their mixed tone and trouble keeping a position, they may not be able to hold onto objects (such as a toothbrush or pencil). About 25-40% of all people with CP have athetoid CP. The damage occurs to the extrapyramidal motor system and/or pyramidal tract and to the basal ganglia.

Incidence and Prevalence

In the industrialized world, the incidence of cerebral palsy is about 2 per 1000 live births. The incidence is higher in males than in females; the Surveillance of Cerebral Palsy in Europe (SCPE) reports a M:F ratio of 1.33:1. Variances in reported rates of incidence across different geographical areas in industrialized countries are thought to be caused primarily by discrepancies in the criteria used for inclusion and exclusion. When such discrepancies are taken into account in comparing two or more registers of subjects with cerebral palsy (for example, the extent to which children with mild cerebral palsy are included), the incidence rates converge toward the average rate of 2:1000. In the United States, approximately 10,000 infants and babies are diagnosed with CP each year, and 1200-1500 are diagnosed at preschool age.

Overall, advances in care of pregnant mothers and their babies have not resulted in a noticeable decrease in CP. This is generally attributed to medical advances in areas related to the care of premature babies that results in a greater survival rate. The incidence of CP increases with premature or very low-weight babies regardless of the quality of care, and the incidence in very premature infants is 6:1000.

Prevalence of cerebral palsy is best calculated around the school entry age of about six years, the prevalence in the U.S. is estimated to be 2.4 out of 1000 children.

Prognosis

CP is not a progressive disorder (meaning the actual brain damage does not worsen), but the symptoms can become worse over time due to ‘wear and tear.’ A person with the disorder may improve somewhat during childhood if he or she receives extensive care from specialists, but once bones and musculature become more established, orthopedic surgery may be required for fundamental improvement. People who have CP tend to develop arthritis at a younger age than normal because of the pressure placed on joints by excessively toned and stiff muscles.

The full intellectual potential of a child born with CP will often not be known until the child starts school. People with CP are more likely to have some type of learning disability, but this is unrelated to a person's intellect or IQ level. Intellectual level among people with CP varies from genius to mentally retarded, as it does in the general population. In most cases persons with CP can expect to have a normal life expectancy; survival is understood to be associated with the ability to ambulate, roll, and self-feed.

Treatment

There is no cure for CP. However, various forms of therapy can help a person with the disorder to function and live more effectively. In general, the earlier treatment begins the better chance children have of overcoming developmental disabilities or learning new ways to accomplish the tasks that challenge them. The earliest proven intervention occurs during the infant's recovery in the neonatal intensive care unit. Treatment may include one or more of the following: physical therapy; occupational therapy; speech therapy; drugs to control seizures, alleviate pain, or relax muscle spasms (e.g. benzodiazepines, baclofen and intrathecal phenol/baclofen); hyperbaric oxygen; the use of Botox to relax contracting muscles; surgery to correct anatomical abnormalities or release tight muscles; braces and other orthotic devices; wheelchairs and rolling walkers; and communication aids such as computers with attached voice synthesizers.

Physical therapy (PT) programs are designed to encourage the subject to build a strength base for improved gait and volitional movement, together with stretching programs to limit contractures. Many experts believe that life-long physical therapy is crucial to maintain muscle tone, bone structure, and prevent dislocation of the joints. Similarly, occupational therapy (OT) helps adults and children maximize their function, adapt to their limitations and live as independently as possible. Orthotic devices such as ankle-foot orthoses (AFOs) are often prescribed to minimize gait irregularities. AFOs have been found to improve several measures of ambulation, including reducing energy expenditure and increasing speed and stride length.

Speech therapy helps control the muscles of the mouth and jaw, and helps improve communication. Just as CP can affect the way a person moves their arms and legs, it can also affect the way they move their mouth, face and head. This can make it hard for the person to breathe; talk clearly; and bite, chew and swallow food. Speech therapy often starts before a child begins school and continues throughout the school years.

Ultimately surgery may be required to alleviate the effects of CP. Surgery for people with CP usually involves one or more surgical interventions. Surgical loosening of tight muscles and releasing fixed joints, is most often performed on the hips, knees, hamstrings, and ankles. In rare cases, this surgery may be used for people with stiffness of their elbows, wrists, hands, and fingers. Abnormal twists of the leg bones, i.e. femur (termed femoral anteversion or antetorsion) and tibia (tibial torsion) are secondary complications caused by the spastic muscles generating abnormal forces on the bones, and often results in intoeing (pigeon-toed gait). Surgical straightening of the leg bones may be performed called derotation osteotomy, in which the bone is broken (cut) and then set in the correct alignment. Cutting nerves on the limbs most affected by movements and spasms is called a rhizotomy, which reduces spasms and allows more flexibility and control of the affected limbs and joints.

Botulinum Toxin A (Botox) injections into muscles that are either spastic or have contractures, the aim being to relieve the disability and pain produced by the inappropriately contracting muscle. However, this treatment induces some degree of paralysis.

In addition, various treatment modalities for CP have been used or proposed. Studies have demonstrated improvement in CP symptomology when hyperbaric oxygen therapy is used as a treatment. Nutritional counseling may help when dietary needs are not met because of problems with eating certain foods. Both massage therapy and hatha yoga are designed to help relax tense muscles, strengthen muscles, and keep joints flexible. Hatha yoga breathing exercises are sometimes used to try to prevent lung infections.

Nevertheless, there is only limited benefit from current therapy. Treatment is usually symptomatic and focuses on helping the person to develop as many motor skills as possible or to learn how to compensate for the lack of them. Conventional MR imaging shows evidence of brain injury and/or maldevelopment in 70%-90% of children with cerebral palsy (CP), though its capability to identify specific white matter tract injury is limited. The great variability of white matter lesions in CP already demonstrated by postmortem studies is thought to be one of the reasons why response to treatment is so variable. There is a need for both a better understanding of the etiology of CP; with better understanding of CP there is also needed better approaches to manage CP including and improved approaches to the diagnosis, prognosis, prophylaxis and therapy of the condition.

### SUMMARY OF THE INVENTION

Disclosed for the first time herein is the characterization of specific white matter tract lesions in children with CP associated with periventricular leukomalacia (PVL). As described below our DTI methodology has been able to ascertain damage to specific white matter tracts in the brains of children with CP at a resolution not previously possible. This high resolution view of damaged tract in children with CP secondary to PVL has changed the understanding of the pathophysiology of motor disturbances in CP in that we find that motor disturbances are due primarily to disruption of afferent sensory input into somatosensory cerebral cortex from the thalamus rather than, as we previously thought, to disturbances of efferent fibers carrying information out of the brain's motor cortex. Based on this new view of white matter pathology in CP secondary to CP, it is clear that passive movement of the arms and legs as currently provided in physical and occupational therapies are not expected to provide stimulation to the brain because nerve fibers relaying this information to the somatosensory cortex are disconnected. Rather, given this new more accurate view of pathology in CP, direct stimulation of the somatosensory cortex is required that bypasses damaged thalamocortical afferents. Our invention bypasses damaged afferent input by providing non-invasive focal electrical stimulation of the somatosensory cortex in children with CP supplied by transcranial magnetic stimulation (TMS) over the surface of the scalp.

Focal TMS provided to the somatosensory cortex in subjects with CP or PVL restores normal levels of neuronal electrical activity, which restores the health of neurons and synapses in this area of the brain, and secondarily restores normal activity of adjacent motor cortex that receives direct cortico-cortical fibers from somatosensory cortex. Since our DTI imaging has revealed intact though partially damaged motor fibers carrying efferent fibers from motor cortex, restored activity in somatosensory and motor cortex, along with conventional therapies, is able to correct imbalances in brain cortical electrical circuitry and restore progression of normal motor development so that children with this form of CP can learn to walk and control movements of their arms and legs.

In a preferred embodiment the technique of diffusion tensor imaging (DTI) was used to provide in vivo characterization of specific white matter tract lesions in children with CP associated with periventricular leukomalacia (PVL). It is to be understood that DTI is not crucial to the invention, but the level if resolution and specificity is helpful and avoids the trial and error that would be needed in the absence of such information.

Magnetic Resonance (MR) imaging techniques, including MR imaging (MRI), diffusion-weighted MR imaging (DWI), and diffusion tensor imaging (DTI), have been established as the imaging techniques of choice for initial characterization and follow-up of CP patients. (Srinivasan L, Dutta R, Counsell S J, et al. Quantification of deep gray matter in preterm infants at term-equivalent age using manual volumetry of 3-tesla magnetic resonance images. Pediatrics 2007; 119:759-65; Huppi P S, Barnes P D. Magnetic resonance techniques in the evaluation of the newborn brain. Clin Perinatol 1997; 24:693-723; Counsell S J, Rutherford M A, Cowan F M, et al. Magnetic resonance imaging of preterm brain injury. Arch Dis Child Fetal Neonatal Ed 2003; 88:F269-274; Huppi P S, Amato M. Advanced magnetic resonance imaging techniques in perinatal brain injury. Biol Neonate 2001; 80:7-14; Huppi P S, Inder T E. Magnetic resonance techniques in the evaluation of the perinatal brain: recent advances and future directions. Semin Neonatol 2001; 6:195-210; Barkovich A J. Brain and spine injuries in infancy and childhood. In: Barkovich A J, ed. Pediatric Neuroimaging. Philadelphia: Lippincott Williams & Wilkins; 2000: 181-84)

Typical MR imaging findings in childhood show enlarged ventricular atria and volume loss in periventricular white matter, often associated with T2 and fluid-attenuated inversion recovery (FLAIR) hyperintense signal intensity and, more rarely, with cysts. (Barkovich A J. Brain and spine injuries in infancy and childhood. In: Barkovich A J, ed. Pediatric Neuroimaging. Philadelphia: Lippincott Williams & Wilkins; 2000: 181-84)

Previously, assessment of injuries to specific white matter tracts has been difficult with conventional MR imaging, as disclosed herein DTI has been used to great effect. Accordingly, 24 children with CP associated with PVL and 35 healthy controls were evaluated with DTI. Criteria for identification of 26 white matter tracts based on 2D DTI color-coded maps were established, and a qualitative scoring system, based on visual inspection of the tracts in comparison with age-matched controls, was used to grade the severity of abnormalities. An ordinal grading system (0=normal, 1=abnormal, 2=severely abnormal or absent) was used to score each white matter tract.

There was marked variability in white matter injury pattern in subjects with PVL, with the most frequent injury(s) to the retrolenticular part of the internal capsule, posterior thalamic radiation, superior corona radiata, and commissural fibers.

DTI was successfully used for in vivo assessments of specific white matter lesions in subjects with PVL and, thus, is a valuable diagnostic tool. The tract-specific evaluation revealed a family of tracts that are highly susceptible in PVL. These important data are relied on and used to tailor treatment options.

The present invention comprises treating subjects with CP or PVL, with specific regard to the sensory deficits that are shown herein to be present in CP/PVL pathology. In a particular embodiment, transcranial magnetic stimulation (TMS) is used to focally and noninvasively stimulate areas of the sensory/somatosensory cortex that are otherwise not receiving normal sensory inputs from, e.g., the thalamic region of the brain. The abnormal inputs can comprise a paucity of normal inputs and/or abnormal conduction. By focally and noninvasively stimulating to sensory areas of the brains of subjects with CP/PVL it is possible to bypass area of pathology and thus alleviate many effects of CP or PVL, for example contractures, spasms, impaired coordination, and atrophy of otherwise underused and/or understimulated motor nerve pathway fibers.

The invention comprises a method for stimulating nerve tissue in a sensory area of the brain of a subject with cerebral palsy or PVL of: determining that an afferent tract to a brain cortex sensory area of the subject manifests pathology; identifying a sensory area of the subject's brain that receives information from the pathologic afferent tract; administering to the subject a modality predicated on the existence of the sensory area that receives deficient afferent information. The determining step can comprise diffusion tensor imaging, determining that the afferent tract provides inadequate or abnormal sensory information. The method can comprise providing stimulation to the specific sensory area of the subject's brain that is missing normal afferent information.

Also disclosed herein is a method for stimulating a sensory cortical area of a subject's brain, that comprises: determining that afferent nerves to a sensory cortical area of the subject's brain manifest pathology; identifying an area of the subject's sensory cortex that corresponds to the afferent nerve pathway; mapping the sensory cortical area to the surface of the subject's head; placing a means for noninvasive focal stimulation of internal nerve tissue at the mapped area of the subject's head; and stimulating noninvasively and focally the area of the sensory cortex that corresponds to pathologic efferent nerves without concomitantly stimulating brain tissue in a generalized manner. The subject can have cerebral palsy or periventricular leukomalacia. The determining step can comprise use of diffusion tensor imaging (DTI). In the method the placing step can comprise placing transcranial magnetic stimulation coils; and, the stimulating step comprises stimulating the area of the sensory cortex with transcranial magnetic stimulation.

Also disclosed is a method for eliciting efferent stimulation of motor fiber nerve tracts in subjects with cerebral palsy that comprises: identifying a sensory area of the subject's brain that has a deficit; mapping the sensory area to the surface of the subject's head; placing a means for noninvasive focal stimulation of internal nerve tissue at the mapped area of the subject's head; stimulating noninvasively and focally the sensory area of the subject's brain that has a deficit without concomitantly stimulating brain tissue in a generalized manner, and eliciting from the area sensory area efferent stimuli along motor fibers. The method can minimize atrophy of the motor fibers. The method can comprise use of diffusion tensor imaging (DTI).

Also disclosed herein is an apparatus for use in neurostimulation of a human subject's head, the apparatus comprising: a body portion configured to fit about the upper portion of the subject's head, whereby the eyes, nose, mouth and preferably the ears are uncovered; at least one device that upon activation induces noninvasive focal neurostimulation of the subject's brain; means for containing the device is a secure manner, the containing means attached to or formed within the body portion. The body portion can comprise fabric, thermal insulating material, or thermal conductive material. The neurostimulation device can comprises a transcranial magnetic stimulation coil, which can be a figure eight coil. The containing means can be a pocket within the body portion. The containing means can comprise a mechanism for removably attaching the coil to the body portion which is a snap, hook, or Velcro component. The apparatus can also contain a means for cooling the neurostimulation device. The apparatus can also comprise skill-building equipment such as a computer program; computer keyboard; monitor; or equipment that is designed to facilitate movement and exercise of muscles, joints or body parts. A means for entertaining or distracting the subject can also be included with the apparatus which can be a television, gaming device, a device to emit sounds such as music or speech, a rack to hold reading material. The apparatus can also include container for shipping or storing the apparatus and instructions for use of the apparatus, as well as instructions for use of the apparatus that are located on the container, in the container, or on the apparatus itself.

Disclosed herein is a method for evaluating the brain of a subject with cerebral palsy comprising: imaging one or more afferent tracts to a brain cortex sensory area of the subject; determining that an afferent tract to a brain cortex sensory area of the subject manifests pathology; and whereby the existence of the pathologic afferent tract indicates that the subject has a sensory deficit that contributes to the subject's symptoms. The determining step can comprise diffusion tensor imaging, can determine that the afferent tract provides inadequate or abnormal sensory information.

A method for designing treatment of a subject with cerebral palsy is disclosed herein, the designing method includes steps if the evaluating method as well as a step of devising a therapy that alleviates symptoms caused by the pathologic afferent tract. The designing method can comprise identifying a specific sensory cortex area of the subject's brain that receives information from the pathologic afferent tract; and, the devising step thereof comprises tailoring the therapy to utilize the specific sensory cortex area of the subject's brain. The devised therapy can provides stimulation to the specific sensory area of the subject's brain that is missing normal afferent information.

Other features and advantages of the invention will be apparent from the detailed description, and from the claims.

Definitions

“Atrophy” indicates a condition of wasting away or diminution in the size of a cell, tissue, organ or part.

By “cell substrate” is meant the cellular or acellular material (e.g., extracellular matrix, polypeptides, peptides, or other molecular components) that is in contact with the cell.

By “control” is meant a standard or reference condition.

“Cortical motor threshold” or “CMT” an energy level that is the energy needed to elicit movement of the fingers when the motor cortex is stimulated for subject. Accordingly, for TMS the level of energy in Joules needed over the motor cortex in order to stimulate finger movement is known in the field as the cortical motor threshold. TMS is generally provided at a level of 90% of the CMT to avoid eliciting unwanted motor movements during therapy.

“Diffusion tensor imaging” or “DTI” is a modality that uses diffusion weighted sequences that are sensitive to the movement of protons fluid. Since axons and their myelin coverings in white matter run lengthwise next to each other water molecules diffuse easily in the direction parallel to their length, but are unable to diffuse freely at right angles to them. Presently, imaging sequences used in DTI can detect the diffusion of water in 6-32 directions in each voxel (cube) of tissue, many more than used in conventional diffusion weighted imaging, making it possible to resolve small changes in the direction of fibers and create detailed maps through a process called tractography. DTI data can also used to calculate objective values for various variables such as “fractional anisotropy” (FA), apparent diffusion coefficient (ADC) or the directionally averaged mean diffusivity (Dav).

By “disease” is meant any condition or disorder that damages or interferes with the normal function of a cell, tissue, organ or subject.

By “effective amount” is meant the amount of an agent or modality required to ameliorate the symptoms of a disease relative to an untreated subject or patient. An effective amount of an active therapeutic agent used to practice the present invention for the treatment of a disease or injury varies depending upon the manner of administration, the age, body weight, and general health of the subject. Ultimately, the attending clinician will decide the appropriate amount and dosage regimen based on judgment parameters know in the art.

“Fractional anisotropy” (FA) is calculated from DTI data and has values ranging from 0 to 1. A value of “0” indicates free movement of water in all directions in the shape of a sphere (isotropic diffusion), and “1” describes the state in which diffusion is restricted within the shape of a cylinder (anisotropic diffusion). FA values close to “1” in white matter pathways indicate predominant movement of water in an ellipsoid space parallel to axons. These high values suggest greater integrity or organization within the white matter, while low FA values suggest damage, necrosis, paucity or immaturity of white matter.

“ICD” refers to The International Statistical Classification of Diseases and Related Health Problems (most commonly known by the abbreviation ICD) provides codes to classify diseases and a wide variety of signs, symptoms, abnormal findings, complaints, social circumstances and external causes of injury or disease. Every health condition can be assigned to a unique category and given a code, up to six characters long. Such categories can include a set of similar diseases. The International Classification of Diseases is published by the World Health Organization. The ICD is used world-wide for morbidity and mortality statistics, reimbursement systems and automated decision support in medicine. This system is designed to promote international comparability in the collection, processing, classification, and presentation of these statistics. The ICD is a core classification of the WHO Family of International Classifications (WHO-FIC).

By “modifies” is meant alters. In the context of the invention, an agent that modifies a cell, substrate, or cellular environment produces a biochemical alteration in a component (e.g., polypeptide, nucleotide, or molecular component) of the cell, substrate, or cellular environment.

The terms “neural plasticity” or plasticity” indicate the use dependent enduring change in neural structure and or function. Neural plasticity is an inherent aspect of maturation and it can also be induced.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

As used herein, a “prodrug” is a pharmacologically inactive compound that is converted into a pharmacologically active agent by a metabolic transformation.

By “subject” is meant a mammal, including, but not limited to, a human or non-human mammal, such as a bovine, equine, canine, ovine, or feline.

By “therapeutic delivery device” is meant any device that provides for the release of a therapeutic agent. Exemplary therapeutic delivery devices include osmotic pumps, indwelling catheters, and sustained-release biomaterials.

“Transcranial Magnetic Stimulation” or “TMS” is noninvasive energy provided through the cranium by a magnetic stimulator, generally delivered a monophasic or biphasic wave form, preferably with maximal energy of 720 Joules at 1.5 or 2 Tesla.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

By “variant” is meant an agent having structural homology to a reference agent but varying from the reference in its biological activity. Variants provided by the invention include optimized amino acid and nucleic acid sequences that are selected using the methods described herein as having one or more desirable characteristics.

### BRIEF DESCRIPTION OF THE DRAWINGS

FIG. 1. A-C, Description of the white matter tract identification protocol. Representative color-coded map or MPRAGE images are shown along with localization in a reference image.

FIG. 2. A-B, Examples of the scoring system obtained from a 7-year-old healthy control (score 0) and patients with PVL (scores 1 and 2). See FIG. 1 for abbreviations.

FIG. 3. Histogram of frequency of scores for individual tracts. A, tracts predominately related to motor pathways; B, tracts predominately related to sensory motor pathways; and C, association and commissural fibers. L indicates left; R, right; CPT/CST, corticopontine/corticospinal tracts; PLIC, posterior limb of the internal capsule; CP, cerebral peduncles; RLIC, retrolenticular part of the internal capsule; PTR, posterior thalamic radiation; SCR, superior corona radiata; IFO/ILF, inferior fronto-occipital/inferior longitudinal fasciculi; SLF, superior longitudinal fasciculus; CC-genu, corpus callosum-genu; CC-body, corpus callosum-body; CC-splenium, corpus callosum-splenium.

FIG. 4. Examples of fiber tracking in three age-matched children. AP indicates anteroposterior.

### DETAILED DESCRIPTION OF THE INVENTION

Although injury to the corticospinal tracts, which carry neuronal impulses from the motor cortex out of the brain (efferent) to the brainstem and spinal cord, has heretofore been thought to be the major determinant of motor impairment in children with PVL, it is shown herein that sensory pathways, including the posterior thalamic radiation that carry impulses into (afferent) the somatosensory cortex of the brain, are affected instead (or concurrently) and more severely than corticospinal. The present invention comprises this new knowledge of neuropathology in CP; in one aspect the invention provides electrical activity in the understimulated, dormant and possibly atrophic somatosensory cortex using non-invasive focal stimulation, such as with TMS.

Despite a wide range of medical interventions in children with CP, there is significant variability in outcome related in part to the heterogeneous nature of the underlying brain pathology. (Maenpaa H, Salokorpi T, Jaakkola R, et al. Follow-up of children with cerebral palsy after selective posterior rhizotomy with intensive physiotherapy or physiotherapy alone. Neuropediatrics 2003; 34:67-71; Chang C H, Albarracin J P, Lipton G E, et al. Long-term follow-up of surgery for equinovarus foot deformity in children with cerebral palsy. J Pediatr Orthop 2002; 22:792-99; Bartlett D J, Palisano R J. Physical therapists' perceptions of factors influencing the acquisition of motor abilities of children with cerebral palsy: implications for clinical reasoning. Phys Ther 2002; 82:237-48; Krach L E. Pharmacotherapy of spasticity: oral medications and intrathecal baclofen. J Child Neurol 2001; 16:31-36).

This outcome data is indicative of the complexity of white matter involvement in PVL. (Fan G G, Yu B, Quan S M, et al. Potential of diffusion tensor MRI in the assessment of periventricular leukomalacia. Clin Radiol 2006; 61:358-64; Anjari M, Srinivasan L, Allsop J M, et al. Diffusion tensor imaging with tract-based spatial statistics reveals local white matter abnormalities in preterm infants. Neuroimage 2007; 35:1021-27. Epub 2007 Feb. 8.)

In one aspect, the present invention demonstrated the utility of DTI to characterize injury in specific white matter tracts in children with PVL, a capability beyond that possible on conventional MR imaging. (Huppi P S, Murphy B, Maier S E, et al. Microstructural brain development after perinatal cerebral white matter injury assessed by diffusion tensor magnetic resonance imaging. Pediatrics 2001; 107:455-60; Hoon A H Jr., Lawrie W T Jr., Melhem E R, et al. Diffusion tensor imaging of periventricular leukomalacia shows affected sensory cortex white matter pathways. Neurology 2002; 59:752-56; Fan G G, Yu B, Quan S M, et al. Potential of diffusion tensor MRI in the assessment of periventricular leukomalacia. Clin Radiol 2006; 61:358-64; Arzoumanian Y, Mirmiran M, Barnes P D, et al. Diffusion tensor brain imaging findings at term-equivalent age may predict neurologic abnormalities in low birth weight preterm infants. AJNR Am J Neuroradiol 2003; 24:1646-53)

As set forth herein, DT-generated color-coded maps were used to classify the status of major white matter tracts by using a 3-grade system based on a qualitative visual assessment of each individual white matter tract. The color-coded map, combined with conventional T1-weighted images, allowed detailed assessment of white matter anatomy of the subject patients. Our results confirmed that, there were deficits in communication between sensory fibers from the thalamus to the sensory cortex. Moreover, even if the motor cortex was injured, sensory cortex was always injured more.

In another aspect, the present invention comprises treating CP or PVL patients with specific regard to the sensory deficits that are shown herein to be present in CP or PVL pathology. In a particular embodiment, transcranial magnetic stimulation (TMS) is used to focally and noninvasively stimulate areas of the sensory/somatosensory cortex that are otherwise not receiving normal sensory inputs from, e.g., the thalamic region of the brain. By focally and noninvasively stimulating to sensory areas of the brains of CP or PVL patients it is possible to alleviate many effects of CP or PVL, for example contractures, spasms, impaired coordination, and atrophy of otherwise understimulation motor nerve pathway fibers.

Diffusion Tensor Imaging (DTI)

Magnetic resonance imaging (MRI) of the brain has become a valuable tool for determining the cause of cerebral palsy (CP) in individual patients as well as for research on the diverse mechanisms that are responsible for its pathogenesis. (Dyet L E, Kenna N, Counsell S J, et al. Natural history of brain lesions in extremely premature infants studied with serial magnetic resonance imaging from birth and neurodevelopmental assessment. Pediatrics. 2006; 118:536-548.) MRI is far superior to other forms of brain imaging, such as computerized axial tomography (CAT) scanning, for evaluating patients with CP because it dramatically enhances the contrast between white matter and gray matter.

Recent advances in an MR technique called diffusion tensor imaging (DTI) are making it possible to evaluate lesions in specific white matter tracts in the brain as well as providing objective, quantitative data on their physical integrity. (Nagae L M, Hoon A H, Stashinko E. Diffusion tensor imaging in children with periventricular leukomalacia: variability of injuries to white matter tracts. AJNR Am J Neuroradiol 2007; 28:1213-1222.)

DTI uses diffusion weighted sequences that are sensitive to the movement of protons in brain water, similar to those used clinically for the rapid diagnosis of stroke and edema. Since axons and their myelin coverings in white matter run lengthwise next to each other like wires in a cable, water molecules diffuse easily in the direction parallel to their length, but are unable to diffuse freely at right angles to them. The imaging sequences used in DTI can detect the diffusion of water in 6-32 directions in each voxel (cube) of tissue, many more than used in conventional diffusion weighted imaging, and this makes it possible to resolve small changes in the direction of fibers and create detailed maps through a process called tractography.

DTI data can also used to calculate numerical variables that describe water diffusion in each voxel within individual white matter pathways. The degree to which water is restricted in its movement by anatomical structures is described by the term “fractional anisotropy” (FA), which has values ranging from 0 to 1. A value of “0” indicates free movement of water in all directions in the shape of a sphere (isotropic diffusion), and “1” describes the state in which diffusion is restricted within the shape of a cylinder (anisotropic diffusion). FA values close to “1” in white matter pathways indicate predominant movement of water in an ellipsoid space parallel to axons. These high values suggest greater integrity or organization within the white matter, while low FA values suggest damage or immaturity of white matter.

The average freedom of diffusion of water molecules in each voxel can also be calculated as the apparent diffusion coefficient (ADC) or the directionally averaged mean diffusivity (Dav). These methods have been used to examine normal white matter development (Huang H, Zhang J, Wakana S, et al. White matter and gray matter development in human fetal, newborn and pediatric brains. Neuroimage 2006; 33:27-38) as well as to assess abnormalities in preterm infants (Huppi P S, Murphy B, Maier S E, et al. Microstructural brain development after perinatal cerebral white matter injury assessed by diffusion tensor magnetic resonance imaging. Pediatrics 2001; 107:455-460) and older patients with cerebral palsy (Thomas B, Eyssen M, Peeters R, et al. Quantitative diffusion tensor imaging in cerebral palsy due to periventricular white matter injury. Brain 2005; 128:2562-2577).

Recently fiber-tracking techniques have been used to predict degree of neurologic impairment for periventricular leukomalacia. For example, DTI fiber-tracking methods have been used to measure FA in the corticospinal tract in a group of 10 infants. These children were born preterm with documented episodes of hypoxia, and all had imaging findings consistent with PVL. However, when evaluations of motor function were performed at 15-63 months of age, five were judged to have spastic diplegia or quadriplegia and half did not have CP.

It has been found that he children with CP tended to have FA values less than 0.5 while those with less disability had values greater than 0.5, suggesting that a cut-off value for FA of <0.5 may be useful for predicting PVL severe enough to produce a severe motor disability. They found that estimation of FA using the fiber tracking method to identify the corticospinal tract was more sensitive to differences in motor outcome than using a region of interest based on anatomical landmarks. Although the study is based on a small group of patients, it suggests that DTI imaging with tractography might be useful for determining prognosis and need for early intervention. The data are consistent with several other recent reports on the relationship between early DTI data and outcome. For example, Arzoumanian et al, and Drobyshevsky et al reported that low FA and high ADC values in white matter using region of interest measurements was associated with motor impairment in a group of premature infants at two years of age (Arzoumanian Y, Mirmiran P D, Barnes K, et al. Diffusion tensor brain imaging findings at term-quivalent age may predict neurologic abnormalities in low birth weight preterm infants. AJNR Am J Neuroradiol 2003; 24:1646-1653; Drobyshevsky A, Bregman J, Storey P, et al. Serial diffusion tensor imaging detects white matter changes that correlate with motor outcome in premature infants. Dev Neurosci 2007; 29:289-301).

Yung et al also found that whole brain white matter volume and reduced FA values were associated with impaired cognitive outcome (Yung A, Poon G, Qui D Q, et al. White matter volume and anisotropy in preterm children: a pilot study of neurocognitive correlates. Pediatr Res 2007; 61:732-736). Krishnan et al found that there was a negative correlation mean ADC in white matter of preterm infants and developmental quotient at two years corrected age (Krishnan M L, Dyet L E, Boardman J P, et al. Relationship between white matter apparent diffusion coefficients in preterm infants at term-equivalent age and developmental outcome at 2 years. Pediatrics 2007; 120:e604-e609). These findings indicate the value that DTI imaging of white matter has for developmental follow-up assessments. Accordingly, a way of monitoring the success of methods of the invention is to monitor FA parameters over time.

DTI has been well studied in normal brain development (Neil J J, Shiran S I, McKinstry R C, et al. Normal brain in human newborns: apparent diffusion coefficient and diffusion anisotropy measured by using diffusion tensor MR imaging. Radiology 1998; 209:57-66; Mukherjee P, Miller J H, Shimony J S, et al. Diffusion-tensor MR imaging of gray and white matter development during normal human brain maturation. AJNR Am J Neuroradiol 2002; 23:1445-56; Mukherjee P, Miller J H, Shimony J S, et al. Normal brain maturation during childhood: developmental trends characterized with diffusion-tensor MR imaging. Radiology 2001; 221:349-58)

DTI has been shown to improve detection of lesions in the first years of life. (Neil J, Miller J, Mukherjee P, et al. Diffusion tensor imaging of normal and injured developing human brain: a technical review. NMR Biomed 2002; 15:543-52; McKinstry R C, Miller J H, Snyder A Z, et al. A prospective, longitudinal diffusion tensor imaging study of brain injury in newborns. Neurology 2002; 59:824-33; Miller S P, Vigneron D B, Henry R G, et al. Serial quantitative diffusion tensor MRI of the premature brain: development in newborns with and without injury. J Magn Reson Imaging 2002; 16:621-32)

Results from DTI studies have provided further understanding of pathogenesis and treatment in a range of neurologic disorders by providing visualization of specific white matter fiber tracts; see review by Horsfield and Jones and others. (Horsfield M A, Jones D K. Applications of diffusion-weighted and diffusion tensor MRI to white matter diseases: a review. NMR Biomed 2002; 15:570-77; Huppi P S, Murphy B, Maier S E, et al. Microstructural brain development after perinatal cerebral white matter injury assessed by diffusion tensor magnetic resonance imaging. Pediatrics 2001; 107:455-60; Hoon A H Jr., Belsito K M, Nagae-Poetscher L M. Neuroimaging in spasticity and movement disorders. J Child Neurol 2003; 18(suppl 1): S25-39; Glenn O A, Henry R G, Berman J I, et al. DTI-based three-dimensional tractography detects differences in the pyramidal tracts of infants and children with congenital hemiparesis. J Magn Reson Imaging 2003; 18:641-48; Lee Z I, Byun W M, Jang S H, et al. Diffusion tensor magnetic resonance imaging of microstructural abnormalities in children with brain injury. Am J Phys Med Rehabil 2003; 82:556-59; Thomas B, Elyssen M, Peeters R, et al. Quantitative diffusion tensor imaging in cerebral palsy due to periventricular white matter imaging. Brain 2005; 128:2562-77)

DTI Imaging Protocol

Data were obtained at a 1.5 T scanner (ACS-NT; Philips Medical Systems, Best, the Netherlands). Initially, all subjects had routine clinical pulse sequences, including sagittal (4-mm section thickness, 1-mm intersection gap) and axial (4-mm section thickness, no intersection gap) T1-weighted (TR/TE, 297.07-598.87/10.5-13 ms), fat-saturated axial T2-weighted (TR/TE, 3992.36-4524.67/110 ms), and FLAIR (TR/TI/TE, 6000/2000/120 ms) sequences.

DTI was acquired following the clinical sequences and consisted of a diffusion-weighted spin-echo pulse sequence with a single-shot echo-planar imaging readout with TR ranging from 6.2 to 9.4 seconds and TE of 80 ms. Fifty axial sections parallel to the anterior/posterior commissure line were acquired, covering the entire brain. The maximal b-value was 700 seconds/mm2, used in a 30 different gradient-direction scheme along with five reference images with minimal diffusion-weighting.(Jones D K, Horsfield M A, Simmons A. Optimal strategies for measuring diffusion in anisotropic systems by magnetic resonance imaging. Magn Reson Med 1999; 42:515-25)

Spin-echo acquisition and sensitivity encoding (SENSE) was used, with an 8-element phased-array coil, converted to a 6-channel coil to be compatible with a 6-channel receiver, with a SENSE reduction factor (R) of 2.5. FOV was adjusted to the brain size, and the imaging matrix was changed within a range of 80×80 to 96×96, resulting in in-plane imaging resolution of 2.0-2.5 mm. All images were zero-filled to a 256×256 matrix. Section thickness was set to approximately the same as that in the in-plane resolution. Scanning times varied from 4 minutes 18 seconds to 6 minutes 34 seconds per sequence. Three repetitions were performed to increase signal intensity-to-noise ratio.

3D-magnetization-prepared rapid acquisition of gradient echo (MPRAGE) images were also obtained with the same section localization, number, and thickness as well as the same FOV of DTI, TR/TE/flip angle of 6.8-8.8/3.3-3.7 ms/8°, scan duration of 3 minutes, and R=2.5.

Postprocessing of DTI Data

All DTI acquisition datasets were transferred to a workstation and corrected for bulk motion by using the automated imaging registration program. (Woods R P, Cherry S R, Mazziotta J C. Rapid automated algorithm for aligning and reslicing PET images. J Comput Assist Tomogr 1992; 16:620-33) DTI postprocessing was performed by using DtiStudio (free software available at http://cmrm.med.jhmi.edu) and included generation of fractional anisotropy (FA), vector maps, and color-coded maps. (Makris N, Worth A J, Sorensen A G, et al. Morphometry of in vivo human white matter association pathways with diffusion-weighted magnetic resonance imaging. Ann Neurol 1997; 42:951-62; Pierpaoli C, Basser P J. Toward a quantitative assessment of diffusion anisotropy. Magn Reson Med 1996; 36:893-906; Pajevic S, Pierpaoli C. Color schemes to represent the orientation of anisotropic tissues from diffusion tensor data: application to white matter fiber tract mapping in the human brain. Magn Reson Med 1999; 42:526-40)

The processing algorithm used assumed that the eigenvector associated with the largest eigenvalue represented the average main fiber orientation of a particular pixel. In the color-coded maps, colors were assigned according to the vector map as blue representing superior-inferior orientation (through the axial plane); green, anteroposterior orientation; and red, laterolateral orientation. Tracts with oblique angles were represented with the appropriate mixture of these basic colors. Color intensity was scaled proportional to FA values.

By use of automated imaging registration programs such as DTIStudio it is possible to assemble two-dimensional and three-dimensional images of the imaged matter.

White Matter Tract Identification

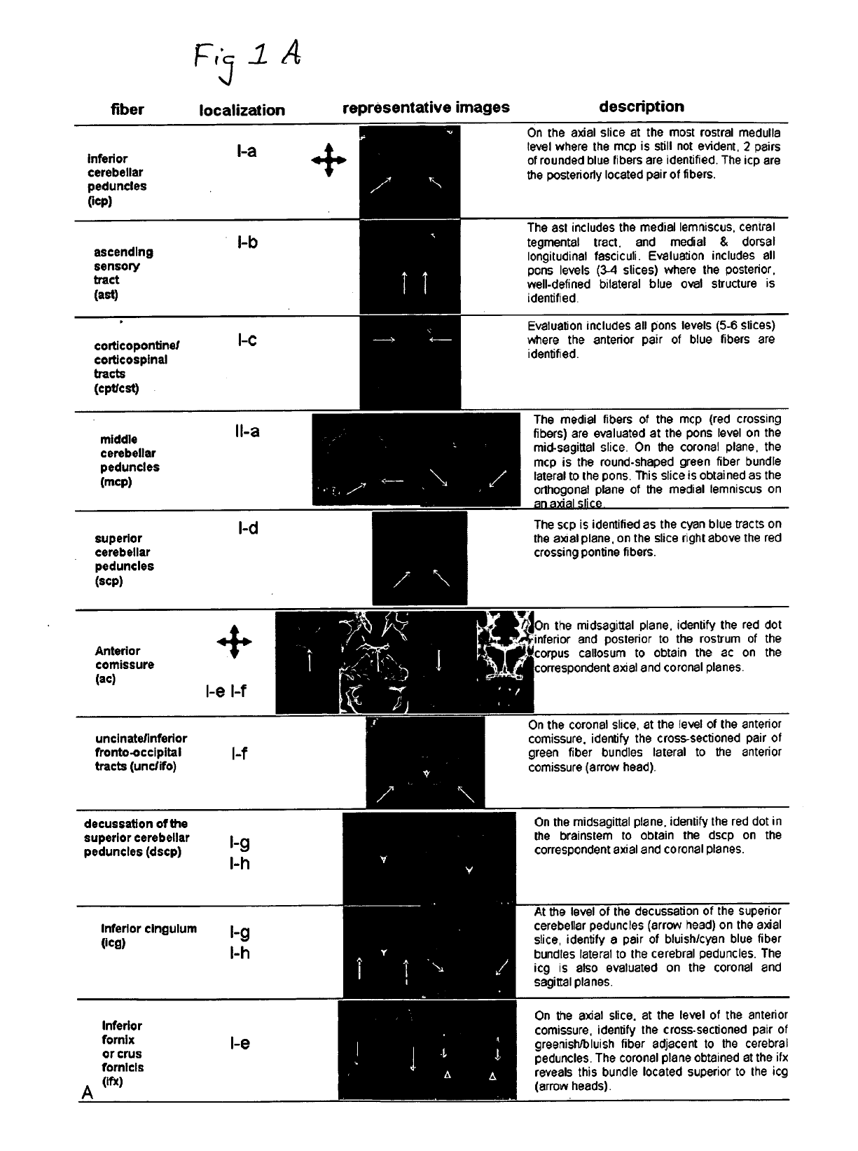

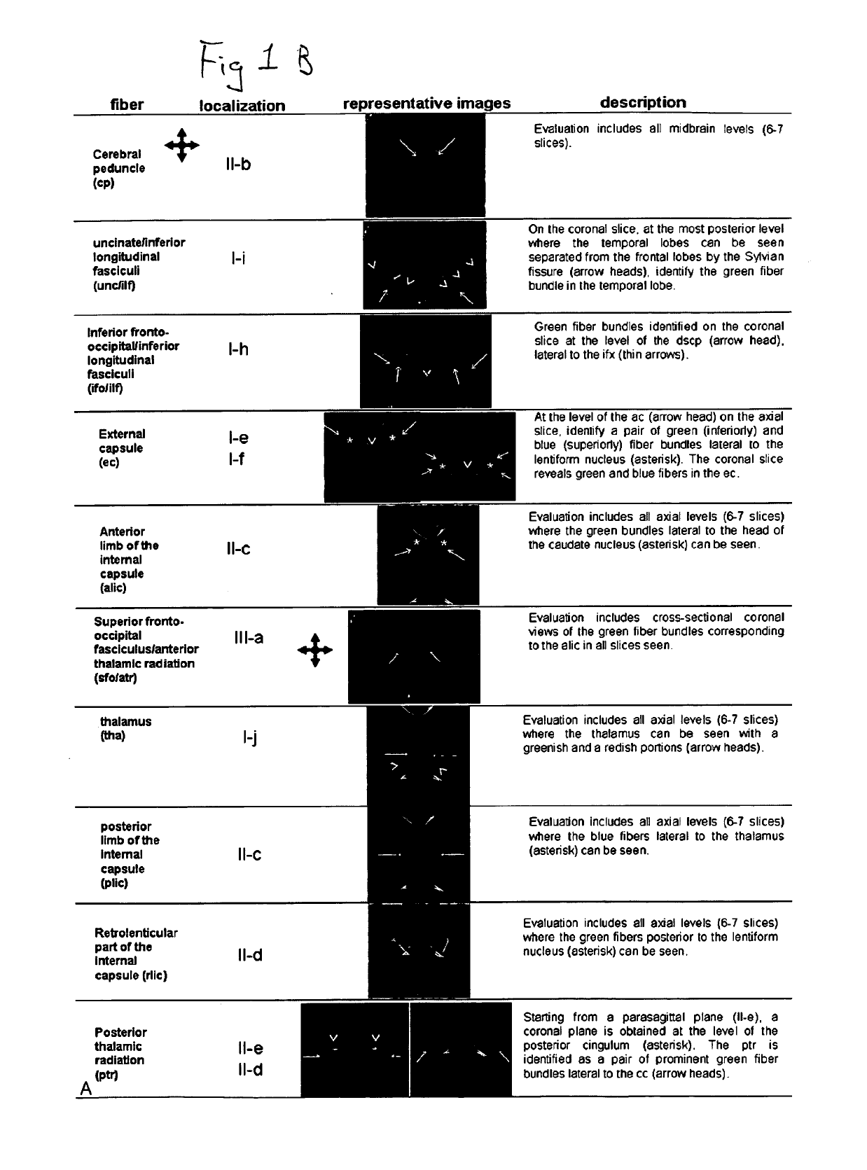

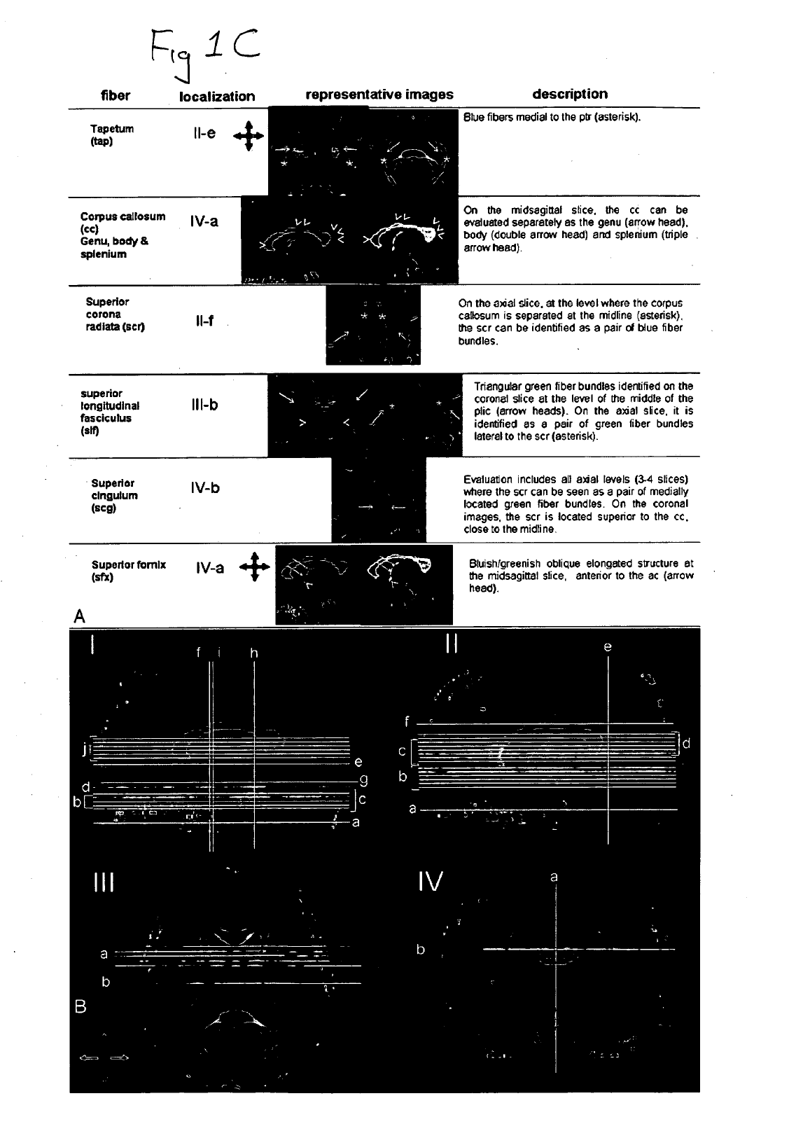

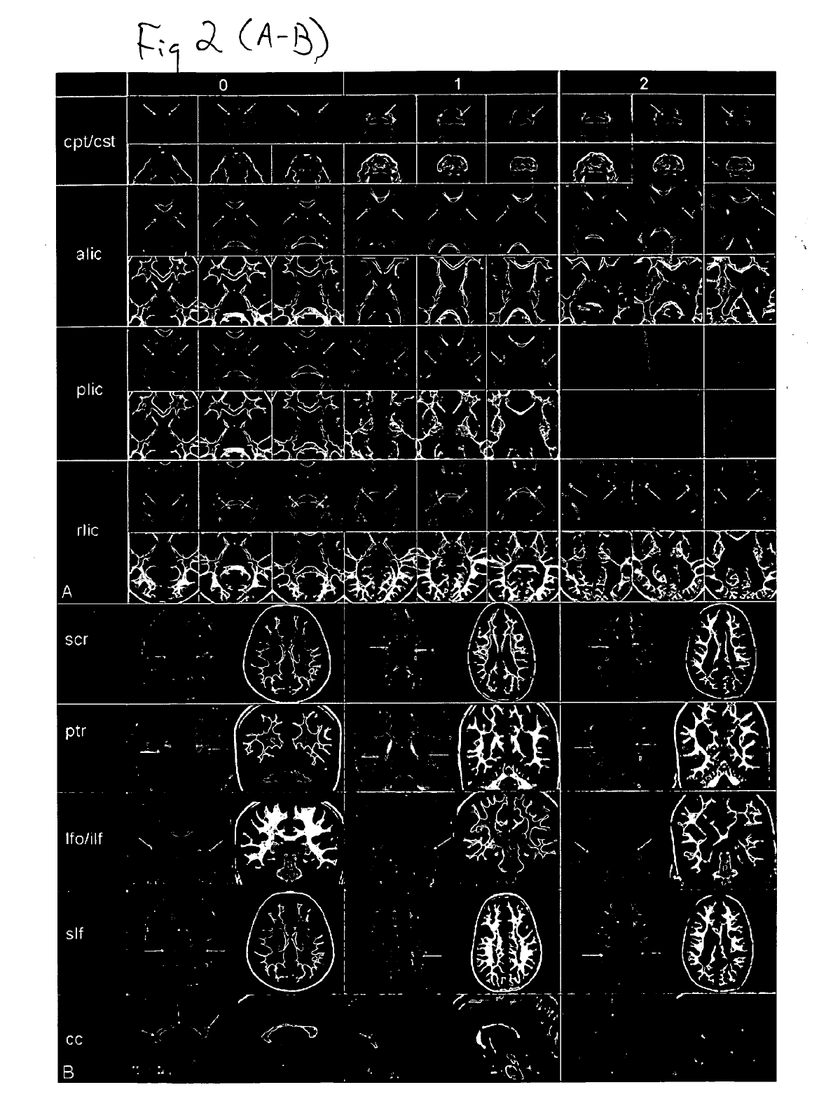

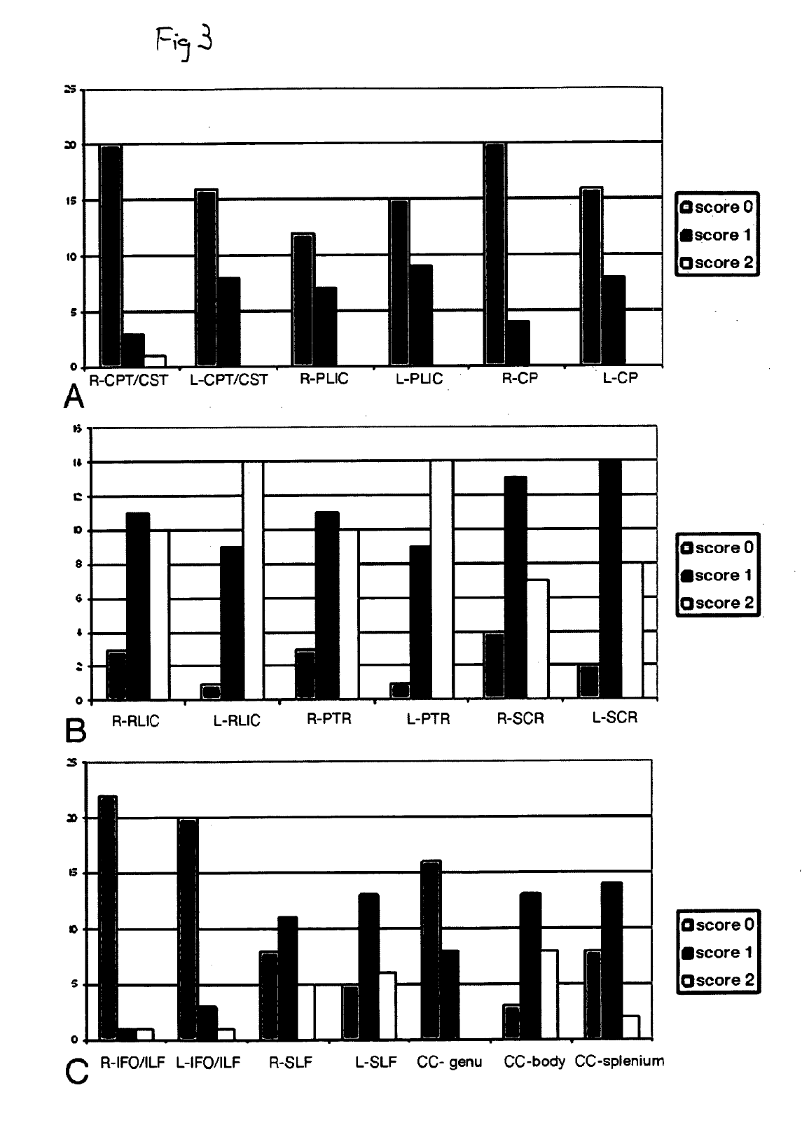

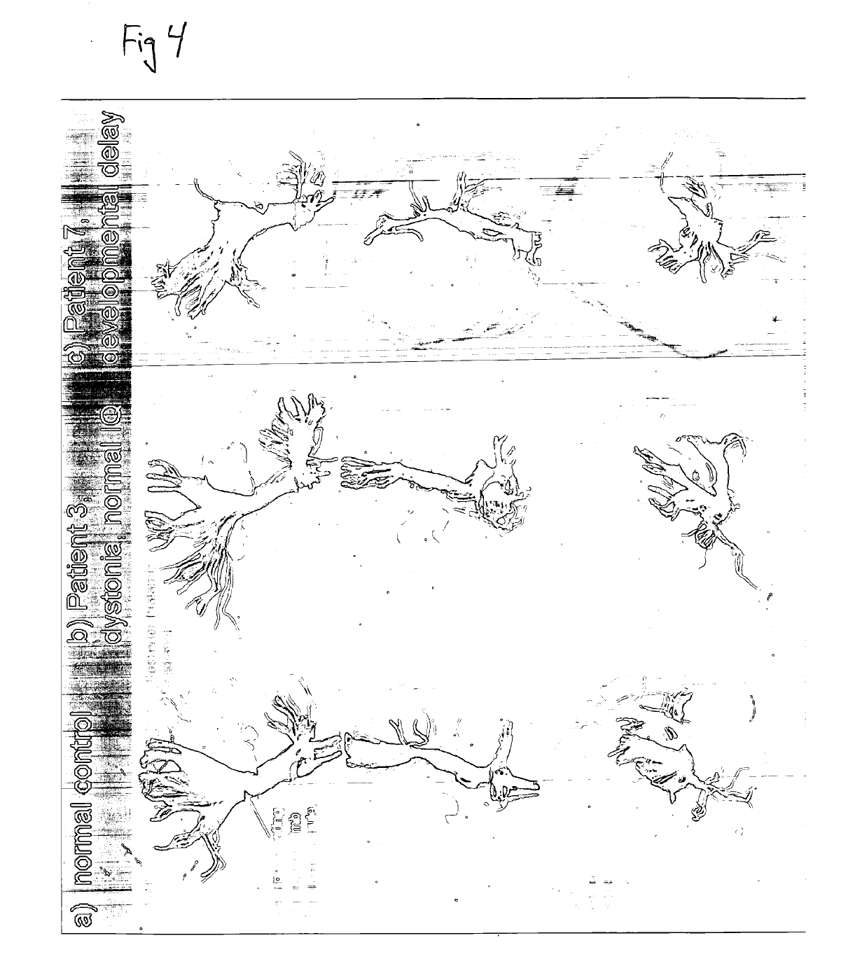

White matter tract identification was performed by using the color-coded maps, with specific criteria listed in FIG. 1. (Pajevic S, Pierpaoli C. Color schemes to represent the orientation of anisotropic tissues from diffusion tensor data: application to white matter fiber tract mapping in the human brain. Magn Reson Med 1999; 42:526-40; Stieltjes B, Kaufmann W E, van Zijl P C, et al. Diffusion tensor imaging and axonal tracking in the human brainstem. Neuroimage 2001; 14:723-35; Wakana S, Jiang H, Nagae-Poetscher L M, et al. Fiber tract-based atlas of human white matter anatomy. Radiology 2004; 230:77-87; Mori S, van Zijl P C. Fiber tracking: principles and strategies—a technical review. NMR Biomed 2002; 15:468-80; Catani M, Howard R J, Pajevic S, et al. Virtual in vivo interactive dissection of white matter fasciculi in the human brain. Neuroimage 2002; 17:77-94)

Two- and 3D representations of some of normal human tracts can be found in Wakana S, Jiang H, Nagae-Poetscher L M, et al. Fiber tract-based atlas of human white matter anatomy. Radiology 2004; 230:77-87.

Although tracts were identified primarily by the color-coded maps, MPRAGE was also used to supplement interpretation, especially for the corpus callosum, anterior commissure at the midsagittal level, and the column and body (superior part) of the fornix. These fiber tracts can be discretely identified by MPRAGE, which offers higher resolution to assess their anatomy.

Structures that benefit the most from identification on color-coded maps include projectional fibers such as the corona radiata, anterior thalamic radiation, sagittal stratum, posterior thalamic radiation, retrolenticular part of the internal capsule, and association fibers such as the superior longitudinal fasciculus, inferior fronto-occipital fasciculus, uncinate fasciculus, and inferior longitudinal fasciculus. These tracts cannot be individually identified on conventional MR imaging because they are intermingled. Color-coded maps, carrying orientation information, can separate individual tracts.

Other structures identified on conventional imaging such as the corticospinal/corticopontine tracts; medial lemniscus; middle, inferior, and superior cerebellar peduncles; and cingulum also benefit from more precise delineation on the color maps. Color maps reveal a range of different colors in the inner architecture of the cerebral peduncles and thalami, showing more details of these structures.

Three Dimensional Pathway Reconstruction

A 3D reconstruction (FIG. 4) was used to visualize the fibers penetrating the posterior limb of the internal capsule and the posterior thalamic radiation. This technique is very powerful to understand visually the 3D trajectory of a tract of interest, its usefulness in routine diagnosis may be more beneficial upon implementation of certain of the following parameters. First, reconstruction is strongly dependent on the location of the reference region of interest used for tracking and on subjective tract editing. Establishing strict protocols for region of interest placement serves to ameliorate these problems. (Stieltjes B, Kaufmann W E, van Zijl P C, et al. Diffusion tensor imaging and axonal tracking in the human brainstem. Neuroimage 2001; 14:723-35; Wakana S, Jiang H, Nagae-Poetscher L M, et al. Fiber tract-based atlas of human white matter anatomy. Radiology 2004; 230:77-87; Mori S, van Zijl P C. Fiber tracking: principles and strategies—a technical review. NMR Biomed 2002; 15:468-80; Mori S, Kaufmann W E, Davatzikos C, et al. Imaging cortical association tracts in the human brain using diffusion-tensor-based axonal tracking. Magn Reson Med 2002; 47:215-23) Such a protocol can be challenging in subjects with PVL, who often present with severe anatomic changes. Furthermore, the reconstruction results are affected by the thresholds (FA and angle) for termination criteria. This effect may be removed by using the same thresholds for all subjects, FA values for the white matter change during brain development,(Mukherjee P, Miller J H, Shimony J S, et al. Normal brain maturation during childhood: developmental trends characterized with diffusion-tensor MR imaging. Radiology 2001; 221:349-58) and the same FA threshold may not result in equivalent reconstruction results for brains with different ages, e.g., in view of the aforementioned partial volume effects. Therefore, use of 3D reconstruction may be limited for visual understanding of severe abnormalities found in color maps, although this becomes less problematic as greater amounts of image data are generated on children of various ages and conditions. For routine, clinical practice, 2D-based examination is an alternative approach, promptly available for straightforward interpretation without extra processing time.

Pathology Scoring of DTI Images

Although qualitative evaluation of images carries a high degree of subjectivity, it reflects the daily activity in neuroradiology. The concept of using MR images of lesions in a scoring system has been shown to add great benefit for classification and follow-up studies. (Liao D, Cooper L, Cai J, et al. Presence and severity of cerebral white matter lesions and hypertension, its treatment, and its control: The ARIC Study—

Atherosclerosis Risk in Communities Study Stroke 1996; 27:2262-70; Loes D J, Hite S, Moser H, et al. Adrenoleukodystrophy: a scoring method for brain MR observations. AJNR Am J Neuroradiol 1994; 15:1761-66; Simon E M, Hevner R, Pinter J D, et al. Assessment of the deep gray nuclei in holoprosencephaly. AJNR Am J Neuroradiol 2000; 21:1955-61)

Images considered as normal in this study were found in a control normative database, including 35 age-matched healthy children from 12 months to 15 years of age. To augment our understanding of normal range, a qualitative grading system including only 3 different grades (0=normal, 1=abnormal, 2=severely abnormal or absent) was adopted as a first approach to more objectively score the white matter tracts.

The present data showed that multiple raters with guidelines can score white matter tracts visualized on DTI reliably. Although experienced neuroradiologists, the two raters participating in the reliability tests were given the criteria described in this study for white matter tract identification and scoring as well as the control database, without formal training. Reducing the scoring to two categories (normal and abnormal) improved agreement across raters and scoring consistency of repeated observations within the primary rater (intrarater agreement).

Using a two-scale grading system shows superior reliability results, and simply distinguishing between normal versus abnormal is important in clinical or research settings, with the understanding that a three-scale grading system can be even more informative when provided by raters which are more experienced with color-coded maps.

Of note, there was variability in the scoring agreement among tracts—several tracts had 100% inter-rater agreement (cerebral peduncles, inferior fronto-occipital/inferior longitudinal fasciculus, and superior fronto-occipital fasciculus). The lowest indexes for both intra- and inter-rater reliability tests performed were seen for the corpus callosum. This could be due to a wide variability in shape and size among different sexes and ages and, perhaps, any individual. This could also indicate that the color-coded maps, enhancing contrast along the edges of the corpus callosum, might have influenced its evaluation. Adding to what was discussed earlier, the corpus callosum might be one opposite case of low specificity to evaluation on color-coded maps.

Data from DTI Imaging

In a previous findings by certain of the present inventors, two patients with PVL were reported whose corticospinal tracts and fibers penetrating the posterior limb of the internal capsule were relatively well preserved, whereas the posterior thalamic radiation was severely affected were reported. (Hoon A H Jr., Lawrie W T Jr., Melhem E R, et al. Diffusion tensor imaging of periventricular leukomalacia shows affected sensory cortex white matter pathways. Neurology 2002; 59:752-56) This was an unexpected observation because the fibers in the corticospinal tract and fibers penetrating the posterior limb of the internal capsule, which are related to motor functions, were expected to be one of the most affected tracts, whereas the posterior thalamic radiation, which connects the thalamus and parietal/occipital lobes and is mostly related to sensory function, was believed to be relatively preserved.

In the current studies, it was observed that both the retrolenticular part of the internal capsule and the posterior thalamic radiation, in which thalamocortical/corticothalamic pathways are the major constituent, were the white matter tracts bearing the most frequent and severe injuries, augmenting our previous report. These results are consistent with a previously reported pattern of lesions in PVL in postmortem data (Okoshi Y, Itoh M, Takashima S. Characteristic neuropathology and plasticity in periventricular leukomalacia. Pediatr Neurol 2001; 25:221-26). To the best of our knowledge, DTI is the first in vivo imaging technique capable of displaying such findings although it is anticipated that addition techniques with comparable or greater capabilities will be developed.

Important constituents of the retrolenticular part of the internal capsule/posterior thalamic radiation tracts, besides the thalamic pathways and the optic radiation, are long corticofugal pathways (most notably parieto-occipito-temporopontine tracts) and cortico-cortical association tracts such as the inferior longitudinal fasciculus and the inferior fronto-occipital fasciculus. Among these fibers, the association fibers were evaluated at different section levels (FIG. 1) and were found to be relatively preserved in most patients.

The pontine tracts pass through the cerebral peduncles and are relayed to the middle cerebellar peduncles (corticopontocerebellar pathway) at the pons. Involvement of the corticopontine tracts in PVL is also possible, but the extent of abnormalities in the cerebral peduncles and the middle cerebellar peduncles was not as severe as that in the retrolenticular part of the internal capsule/posterior thalamic radiation.

It is also noted that the corticospinal tracts were also often affected in this patient population, though the percentage of tracts scored as abnormal was higher for the retrolenticular part of the internal capsule and posterior thalamic radiation tracts than for the corticospinal tract.

Injuries of the commissural fibers were also prevalent in this patient population; this finding agrees with previous MR imaging observations of patients with PVL. (Davatzikos C, Barzi A, Lawrie T, et al. Correlation of corpus callosal morphometry with cognitive and motor function in periventricular leukomalacia. Neuropediatrics 2003; 34:247-52; Baker L L, Stevenson D K, Enzmann D R. End-stage periventricular leukomalacia:

MR evaluation. Radiology 1988; 168:809-15; Flodmark O, Roland E H, Hill A, et al. Periventricular leukomalacia: radiologic diagnosis. Radiology 1987; 162(1 Pt 1):119-24; Flodmark O, Lupton B, Li D, et al. MR imaging of periventricular leukomalacia in childhood. AJR Am J Roentgenol 1989; 152:583-90; Truwit C L, Barkovich A J, Koch T K, et al. Cerebral palsy: MR findings in 40 patients. AJNR Am J Neuroradiol 1992; 13:67-78)

Among the commissure fibers, the splenium of the corpus callosum and tapetum were most severely affected and are believed to contain commissural projections from the parietal, occipital, and temporal lobes. Combined with severe atrophy of the retrolenticular part of the internal capsule/posterior thalamic radiation, these results strongly suggest concentration of white matter injuries in the parietal and occipital white matter. These are non-motor fibers.

One of the definite advantages of DTI is the ability to make objective/quantitative measurements of tract diameters and such tract-specific DTI parameters are complementary to the present protocols. These objective measurements include parameters such as fractional anisotropy (FA), apparent diffusion coefficient (ADC) and the directionally averaged mean diffusivity (Dav). (Thomas B, Elyssen M, Peeters R, et al. Quantitative diffusion tensor imaging in cerebral palsy due to periventricular white matter imaging. Brain 2005; 128:2562-77; Fan G G, Yu B, Quan S M, et al. Potential of diffusion tensor MRI in the assessment of periventricular leukomalacia. Clin Radiol 2006; 61:358-64; Stieltjes B, Kaufmann W E, van Zijl P C, et al. Diffusion tensor imaging and axonal tracking in the human brainstem. Neuroimage 2001; 14:723-35; Xue R, van Zijl P C, Crain B J, et al. In vivo three-dimensional reconstruction of rat brain axonal projections by diffusion tensor imaging. Magn Reson Med 1999; 42:1123-27; Mori S, Kaufmann W E, Davatzikos C, et al. Imaging cortical association tracts in the human brain using diffusion-tensor-based axonal tracking. Magn Reson Med 2002; 47:215-23)

The variability in white matter injury in motor and sensory pathways was clearly demonstrated with DTI. With the increased number of children studied and acquisition of additional control data, this technique will advance understanding of brain injury in children with childhood neurologic disorders, including CP. Our evaluation protocol is expected to be a guideline for routine clinical evaluation of patients with CP. The evaluation results provide clues to understand pathogenesis and may ultimately lead to improvements in clinical classification and treatment for children with CP and other neurologic disorders of childhood by providing specific treatment options based on the pattern of white matter injury.

Stimulation with Transcranial Magnetic Stimulation (TMS)

TMS was introduced in the 1980s, making noninvasive repeated cortical stimulation possible. Repetitive TMS (rTMS) when applied to the brain can induce effects that outlast the stimulation period. Accordingly, neural plasticity emerges as a result of such interventions. The underlying physical principles of magnetic neural stimulation, design of TMS-related equipment, and parameters used in TMS protocols on subjects are addressed in The Oxford Handbook of Transcranial Stimulation, (e.g., Sections I, II, III, and VI, and addresses TMS administration to pediatric subjects, e.g., in chapters 22 and 25) which is full incorporated herein for such purposes (The Oxford Handbook of Transcranial Stimulation, Wasserman et al. eds.; Oxford Univ. Press, 2008). An advantage of performing TMS on patients in need of overcoming a neurological deficit is that the plasticity induced by TMS can augment the age-dependent neural plasticity that may also be present.

TMS is generally provided by a magnetic stimulator that delivers a monophasic or biphasic wave form, presently with maximal energy is 720 Joules at 1.5 or 2 Tesla. TMS coils are placed on either side or both sides of the patient's head to deliver rapid pulses over selected areas of sensory or motor cortex based on white matter pathology seen on the MR DTI imaging. Coils can be held in place over the cortex in a conventional apparatus with the child reclining. In one embodiment the coils are contained within a specially fabricated helmet/cap/headgear.

In one range of embodiments, pulse sequences as slow as 1 Hz or as rapid as 10 Hz are used. Longer sessions of pulsed TMS or different patterns of rapid stimulation and rest over a treatment session can be efficacious, as well as several sessions throughout the day. Infants as young as 6 months and children as old as teenagers are the preferred age range for treatment in accordance with the invention; this age range generally corresponds to the greatest degree of neural plasticity. Additionally, however adults with CP could continue to benefit if TMS therapy was started as child; other adults with CP also benefit and ongoing neural plasticity may benefit accordingly. The value of somatosensory input, which herein can comprise TMS, and the existence of enduring neuroplasticity is shown for example in Celnik et al., “Somatosensory stimulation enhances the effects of training functional hand tasks in patients with chronic stroke” Arch Phys Med Rehabil 80:1369-76 (November 2007).

The TMS therapy in accordance with the invention can be tailored directly to individual patients using semi-quantitative visual analysis of MR images as well as by use of quantitative analysis of the images using such parameters as fractional anisotropy (FA) and mean diffusivity. DTI imaging of white matter and the objective parameters obtained by processing of this information is useful in developmental follow-up assessments. Accordingly, a way of monitoring the success of methods of the invention is to monitor FA parameters over time.

It has been shown that there are correlations between the amount of TMS stimulation needed to achieve a desired result and the quality of the nerves or pathway being stimulated; DTI and FA data are correlated with TMS parameters. Determination of CMT in patients undergoing therapy for white matter disorders in cerebral palsy will also be quite useful since it has recently been determined that CMT is inversely related to fractional anisotropy (FA) of white matter, an indicator of white matter integrity (Kloppel et al, Neuroimage 2008, 40:1782-1791). This means that low FA numbers, which indicate a disorder of white matter, is associated with elevated CMT, or a higher energy level needed to stimulate motor movement. Patients with cerebral palsy have low FA values that correspond to the degree of white matter injury. Young children also have higher CMT's than older children. Accordingly, setting the energy level according to CMT in our patients will allow us to normalize energy levels for both age of patients and degree of white matter injury.

### EXAMPLES

### Example 1

### Selection of Pediatric CP and Control Populations

In this example, 37 children with CP associated with PVL and 35 healthy controls were evaluated with DTI as set forth above. Criteria for identification of 26 white matter tracts based on 2D DTI color-coded maps were established, and a qualitative scoring system, based on visual inspection of the tracts in comparison with age-matched controls, was used to grade the severity of abnormalities. An ordinal grading system (0=normal, 1=abnormal, 2=severely abnormal or absent) was used to score each white matter tract.

In order to evaluate childhood CP, 37 patients with CP were consecutively scanned with DTI. Criteria for enrollment in the study were: 1) aged birth to 18 years, 2) diagnosis of CP, and 3) a clinically indicated brain scan (for diagnosis or follow-up).

In this example we focused on a subsample of 24 children born at fewer than 37 weeks gestation with PVL diagnosed by neuroradiologic review of conventional MR imaging. There were 14 boys and 10 girls in this study group, ranging in age from 16 months to 13 years 3 months, with a mean age of 6 years. Gestational age at birth ranged from 23 to 34 weeks (mean, 29 weeks). Most children had spastic diplegia (18/24, 75%); 3, spastic quadriplegia; 2, hemiplegia; and 1, ataxic CP with hypotonia.

The DTI protocol was preceded by a conventional MR imaging with standard imaging protocol. A neuroradiologist not involved in the present study interpreted the conventional images, which were reviewed with the patients' families. Most patients (32/34) required sedation for the conventional clinical images and remained sedated for the DTI research images.

Normative data for age-matched controls were obtained from 35 children from a pediatric DTI de-identified database (cmrm.med.jhmi.edu). Controls were distributed in the age ranges of 12-23 months (5 toddlers), 2-3 years (11 children), 4-5 years (5 children), 6-8 years (6 children), 10 years (2 children), and 12-15 years (6 teenagers).

### Example 2

### Identification of Fiber Tracks

Criteria for DTI-based identification of various white matter tracts at 26 locations were established and applied to 24 children with CP associated with PVL as well as in a group of 35 unaffected controls to elucidate further the diversity of white matter tract injury involvement in PVL. The qualitative scoring system, based on visual inspection of the white matter tracts, was used to describe the status of the various white matter tracts.

Fiber tracking was performed by using DTIStudio, which uses the fiber-assignment continuous tracking approach. (Mori S, Crain B J, Chacko V P, et al. Three-dimensional tracking of axonal projections in the brain by magnetic resonance imaging. Ann Neurol 1999; 45:265-69) By combining information from FA and vector maps, this approach allows for 2D and 3D reconstruction of fibers in a continuous vector field. The threshold chosen for FA was 0.15 and the angle threshold, 60°. These thresholds were lower than those used in previous studies due to partial volume effects between structures of the brain and the lower FA of white matter in pediatric brains compared with those of adults (Stieltjes B, Kaufmann W E, van Zijl P C, et al. Diffusion tensor imaging and axonal tracking in the human brainstem. Neuroimage 2001; 14:723-35; Mori S, Crain B J, Chacko V P, et al. Three-dimensional tracking of axonal projections in the brain by magnetic resonance imaging. Ann Neurol 1999; 45:265-69). The initial tracking was started from a region of interest drawn on the color-coded orientation maps. A “brute force” approach (Conturo T E, Lori N F, Cull T S, et al. Tracking neuronal fiber pathways in the living human brain. Proc Natl Acad Sci USA 1999; 96:10422-27; Huang H, Zhang J, van Zijl P C, et al. Analysis of noise effects on DTI-based tractography using the brute-force and multi-ROI approach. Magn Reson Med 2004; 52:559-65) was used, in which fiber tracking was initiated from all pixels, and tracking results that penetrated the region of interest were included. A multiple region of interest reference scheme was used, including “AND” and “NOT” operations: “AND” operation, restricting the tracking to only the fibers that penetrate both regions of interest, and “NOT” operation, excluding fibers within the respective region of interest. (Wakana S, Jiang H, Nagae-Poetscher L M, et al. Fiber tract-based atlas of human white matter anatomy. Radiology 2004; 230:77-87; Mori S, van Zijl P C. Fiber tracking: principles and strategies—a technical review. NMR Biomed 2002; 15:468-80)

To demonstrate the variability of white matter tract injury in PVL, we assembled two fiber tracts in three children in 3D. To demonstrate the relatively preserved tracts in the posterior limb of the internal capsule, we drew one region of interest on the posterior limb of the internal capsule, yielding tracking of all the fibers penetrating this structure. To illustrate injury to the posterior thalamic radiation, the major constituent of the retrolenticular part of the internal capsule, which is often severely affected in PVL, we drew two regions of interest. The first region of interest was drawn in the coronal plane, cross-sectioning the retrolenticular part of the internal capsule, and the second region of interest defined the thalamus. An AND operation selected the fibers passing through both regions of interest. For both the posterior limb of the internal capsule and the posterior thalamic radiation reconstruction, fibers that apparently were not related to the tracts of interest, such as the corpus callosum and the anterior limb of the internal capsule, were rejected by using a NOT operation.

### Example 3

### Scoring of Fiber Tracts

Once the tracts were identified on the basis of the protocol described in the previous Example (also see., e.g., FIG. 1.), an evaluation was completed by using all 3 orthogonal planes of the interactive viewer in DTIStudio. An ordinal grading system (0=normal, 1=abnormal, 2=severely abnormal or absent) was used by the primary study rater to score each tract.

Abnormalities of the white matter tracts were based on size reduction on visual inspection in comparison with age-matched controls, in which white matter tracts were all scored 0. The recognition that a significant decrease of diffusion anisotropy could lead to the appearance of a smaller tract size and thus be scored as abnormal was considered in the interpretation. If size reduction of the tract was identified, the tract was scored as abnormal (score 1). A questionable abnormality was conservatively scored as normal. A structure absent or so abnormal that it could hardly be identified was characterized as severely abnormal or absent (score 2).

To assess inter-rater reliability, two experienced neuroradiologists scored the study data independently, masked to clinical information on the patients. The two raters received instructions as described herein regarding the structures to be scored and the control data set. To establish intrarater reliability estimates for the white matter tract grading, the primary rater (L.M.N.) repeated the tract scoring, and observations at times 1 and 2 were compared. Percentage of agreement was used to rate intra- and inter-rater reliability of this grading system.

Scoring Results

In this study sample of children with PVL, 19 tracts were graded as abnormal by using the 0-2 scoring system. The qualitative examination revealed striking differences between the posterior limb of the internal capsule and the retrolenticular part of the internal capsule/posterior thalamic radiation tracts, in terms of the frequency and degree of injuries.

Some examples of affected tracts and of the grading results are shown in FIG. 2, including the corticopontine/corticospinal tracts, posterior limb of the internal capsule, retrolenticular part of the internal capsule, posterior thalamic radiation, inferior fronto-occipital/inferior longitudinal fasciculi, superior longitudinal fasciculus, superior corona radiata, and the corpus callosum. For the posterior limb of the internal capsule, no example for score 2 (most severe) was found in this patient population. Histograms of frequency of scores for the illustrated individual tracts are also shown in FIG. 3.

To depict visually the trajectories of the affected fibers, the white matter tracts were reconstructed in 3D in three 7-year-old children, including 1 healthy control and 2 patients with PVL (FIG. 4). One of the patients (FIG. 4B) displayed relative preservation of the fibers penetrating the posterior limb of the internal capsule (score 0) and reduced fibers in the posterior thalamic radiation (score 1). FIG. 4C shows a second child in whom the posterior thalamic radiation fibers are more severely affected (score 2). In this example, it can be clearly seen that the corona radiata is also affected (score 2).

Other white matter tracts that were frequently affected included the corticopontine/corticospinal tracts and the corpus callosum, whereas association fibers and limbic fibers (fornix [left side: score 1=2 cases (8.3%)] and cingulum [right side: score 1=2 cases (8.3%); left side: score 1=4 cases (16.6%)]) were relatively more preserved. In agreement with the lesions seen in the retrolenticular part of the internal capsule and posterior thalamic radiation, abnormalities of the corpus callosum were most often seen along the body and splenium of the corpus callosum. The tapetum, believed to be a part of temporal commissural fibers, was affected in most patients as well (right side: score 2=16 cases [66%], score 1=4 cases [16.6%], score 0=4 cases [16.6%]; left side: score 2=18 cases [75%], score 1=3 cases [12.5%], score 0=3 cases [12.5%]).

Prominent sensory tracts in the brain stem (medial lemniscus) were all scored 0 in this population of patients (images not shown). The cerebellar peduncles, which include sensory and motor fibers, were affected in 8/24 patients (score 1, 33.3%). Notable was the extent of pathology in connections between the thalamus and the cortex, most strikingly seen in the 3d images.

Percentage agreement was used as a first step to rate inter-rater reliability of this grading system. On a 3-point scale (0, 1, 2) percentage agreement between the 2 additional raters was 78%. Reducing the categories to a 2-point scale (normal/abnormal) improved inter-rater agreement to 84%. Both are acceptable. Percentage scoring agreement between the 2 raters and the primary study rater ranged from 0.68 to 0.73 agreement on the 3-point scale and 0.77-0.79 on a normal/abnormal rating scale.

Intrarater reliability estimates for the primary study rater (observations 1-2) were 86% agreement; intrarater agreement improved to 91% on a 2-point scale (normal 0/abnormal 1). The percentage agreement reported represented an average of comparisons across all white matter tracts scored. In fact, with the 3-point qualitative scoring system, there was 90%-100% agreement among all 3 raters on selected tracts: cerebral peduncles; middle cerebellar peduncles, sagittal view; inferior fronto-occipital/inferior longitudinal fasciculus; superior fronto-occipital fasciculus; posterior limb of the internal capsule; thalamus; uncinate/inferior fronto-occipital fasciculus; and inferior cingulum.

### Example 4

### Stimulation with Transcranial Magnetic Stimulation (TMS)

Prior to TMS administration, the brains of infants and children with cerebral palsy are imaged, e., g., with magnetic resonance imaging and diffusion tensor imaging to determine if they have periventricular leukomalacia (PVL) that disrupts white matter. The areas of motor cortex and somatosensory cortex with disrupted white matter connections are mapped onto the surface of the brain using available methods so that TMS can be targeted to these areas.

The TMS coils are placed over the scalp above cortex with damaged white matter using digitizing methods that monitor the position of the head as well as the location of damaged white matter based on MRI-DTI information. Maximal damage to white matter generally will be within the areas of somatosensory cortex and this area will be the target of maximal TMS stimulation. Targeted areas of cortex are assessed periodically using MR imaging as the child's head grows to create new maps of cortex onto the surface of the head. Specialized TMS-related equipment can be used in pediatric and juvenile patient populations in order to administer effectively focal non-invasive brain stimulation, and to facilitate patient compliance with the routine administration of TMS; patient compliance is quite important in pediatric and juvenile CP populations.

TMS is provided by a magnetic stimulator that delivers a monophasic or biphasic wave form, with maximal energy is 720 Joules at 1.5 or 2 Tesla. TMS coils are placed on either side or both sides of the patient's head to deliver rapid pulses over selected areas of sensory or motor cortex based on white matter pathology seen on the MR DTI imaging. Coils can be held in place over the cortex in a conventional apparatus with the child reclining. In one embodiment the coils are contained within a specially fabricated helmet/cap/headgear.

In one TMS administration embodiment, high frequency TMS sequences are delivered to each side of the head separately in therapy sessions five days per week which last approximately 2 hours each. The TMS part of the sessions will last approximately ½ hour (preparation and actual stimulation session), and then the child will participate in physical therapy and exercise sessions for the remaining 1½ hours. In one embodiment the PT and OT is provided concurrent with TMS stimulation.

In one embodiment for the TMS pulse sessions, initial pulse sequences will be at 5 Hertz (cycles per second) for 1 minute (300 pulses), then a silent period of 2 minutes, followed by another cycle (one minute of 5 Hz, then another silent period of 2 minutes), and so on for a total of 5 minutes of pulses corresponding to approximately 1500 pulses per session. The same sequence can then be delivered to the opposite side of the brain. In one embodiment, lower intensity sequences are used as maintenance after an initial treatment effect has been achieved.

The TMS therapy as described here is preferably tailored directly to individual patient by using semi-quantitative visual analysis of MR images as well as by use of quantitative analysis of the images using such parameters as fractional anisotropy (FA) and mean diffusivity.

The high frequency pulses are preferably delivered at an energy level that is approximately 90% of the energy needed to elicit movement of the fingers when the motor cortex is stimulated for each child. The level of TMS energy in Joules needed over the motor cortex in order to stimulate finger movement is known in the field as the cortical motor threshold (CMT) and is standard practice. Determination of the CMT in these patients will allow energy to be normalized for different ages of patients and other factors such as head size and thickness of skull and scalp tissue. Use of energy levels below the CMT prevents unwanted movements and helps to minimize the incidence of seizures.

This is a general description of the method, it is understood by those of skill in the art that variations will be used to implement optimal treatment parameters for specific ages of children with CP. Patients treated with TMS will have improved ability to walk, use their hands, and avoid contractures. It is known that neural pathways that are underutilized undergo atrophy. Methods in accordance with the present invention serve to avoid atrophy of existing motor pathways. It is also contemplated that methods of the invention will also elicit growth of motor or sensory fibers.

### Example 5

### Treatment of a Patient with Cerebral Palsy by Focal Brain TMS Therapy Directed by DTI Magnetic Resonance Imaging

A two year old boy is evaluated by a neurologist because he is not able to walk and appears to have stiffness in his legs and arms. Normally children begin to walk at about one year to 15 months of age. History elicited from the child's mother indicates that the boy was born prematurely and that other developmental milestones such as his ability to sit up by himself, and feed himself with his fingers are also delayed compared to other children. Examination by the neurologists reveals that the child is unable to sit or stand and he has difficulty holding onto objects placed in his hands. His legs and arms are stiff when the neurologist attempts to flex and extend them at the elbows, wrists, knees and ankles and reflexes elicited by tapping the tendons at these joints are very active. These findings indicate that the boy has muscle spasticity and stiffness in all four extremities, and these problems are more severe in the legs than the arms. Based on these findings as well as the history that the boy was born prematurely, the neurologist makes a working diagnosis that the child has a form of cerebral palsy (CP) called spastic diplegia.

This form of CP is usually caused by damage to the white matter of the brain, also referred to as periventricular leukomalacia (PVL). White matter in the brain transfers information between different parts of the brain. A magnetic resonance image (MRI) of the brain that includes the diffusion tensor imaging (DTI) is performed, making it possible to distinguish between very small white matter pathways or tracts and to determine precisely which pathways have been injured in this patient. Data from this form of MRI scanning is processed using computer software called DTI studio. Comparison of DTI data from this patient with data from a set of normal patients shows that white matter pathways carrying information from the thalamus to the somatosensory cortex in the brain are severely damaged, and that white matter carrying messages from the motor cortex out of the brain to the spinal cord shows mild injury but the pathways remain mostly intact. The quality of white matter input to the somatosensory cortex is determined by visual assessment of white matter tracts (tractography) as well as by determining numerical data for fractional anisotropy (FA), which provides information on the microstructure of the white matter, within specific tracts entering the cortex. DTI imaging is used to map the area of somatosensory cortex that is receiving deficient or defective input from white matter fibers.

This patient's data is consistent with other children with spastic diplegia and PVL where physical disability is related to damage to white matter, and the damage preferentially affects white matter that carries excitatory messages such as proprioception into the brain from the thalamus so they can activate the somatosensory cortex. Without afferent excitatory stimulation from nerve fibers in the white matter carrying information from the thalamus to the cortex, the cortex is inactive and begins to atrophy. Since the somatosensory cortex normally provides excitement to the motor control cortex, the motor control cortex also is less active and begins to atrophy.

The child's parents and the neurologist would like to start a therapeutic program for the child that can lead to acceleration of his motor development. If left untreated, children with this history of prematurity, neurological examination and inability to walk and use their hands at age two are likely to be severely impaired in the future. In the absence of the treatment set forth below, a typical is that he would be confined most of the day to a wheelchair and unable to walk, and to require assistance with feeding himself. This outcome is typical even with physical therapy (for sitting and walking and large muscle groups) and occupational therapy (for skills using the hands) as routinely used for children with CP. These therapies can reduce muscle spasms and improve the flexibility of the joints, but they usually do not result in major improvements such as the ability to walk. This ineffectiveness is related to the fact that the somatosensory cortex in these children is not receiving adequate electrical stimulation from the thalamus, which usually activates it by releasing excitatory neurotransmitters.

In accordance with the present invention, this patient is treated using transcranial magnetic stimulation (TMS) to activate the dormant somatosensory cortex that is lacking its natural source of stimulation in the thalamus. Accordingly, areas of cortex needing stimulation are digitally mapped onto the surface of the child's scalp overlying the cortex. The child is started on daily therapy sessions that include TMS stimulation followed by sessions of physical and occupational therapy.

For the TMS sessions, the energy level delivered by the TMS coil is set at a level that is 90% of the energy in Joules (less than 720 J) needed to stimulate motor activity in the muscles of the fingers, defined as the motor threshold (Kammer, et al, 2001). This energy level is delivered in high frequency (5 Hz) monophasic or biphasic waveforms over the area of somatosensory cortex as determined from DTI imaging data on each side of the brain. In this patient a pulse sequence of one minute of 5 Hz alternating with two minutes of rest is continued until 5 minutes total of TMS and 1500 pulses have been delivered on one side. Then the other side of the brain is stimulated according to the same protocol. The surface area of the somatosensory cortex in young children indicates that TMS coils can focally stimulate the somatosensory cortex selectively independent of the motor cortex. Motor cortex can also be stimulated selectively, or both areas can be stimulated together. In one embodiment the present invention comprises a customized cap designed for each child can be fixated to the head using fiduciary marks on the scalp, whereby accurate focal stimulation can be maintained during focal non-invasive therapy sessions of neurostimulation. The same areas of stimulation can be maintained when the cap is removed and replaced over several months time. The placement of the TMS coils can be monitored before each TMS therapy session using a computerized 3-D model of the child's skull and underlying cortical areas ascertained by MR imaging. As growth takes place, movement of the TMS coils immobilized within the cap with respect to the underlying scalp will be monitored and the size of the cap and position of the TMS coils will be changed as needed.

The TMS is delivered through coils included in a specially designed cap that is customized for the patient in order to place the coils over cortex served by damaged white matter in a reliable and easy to replicate manner. TMS sessions in this patient occur on 7 days per week, and after each focal noninvasive neurostimulation session for approximately 30-120 minutes specific exercises are prescribed to stimulate use of the hands and legs and to facilitate the building of skills. On five days a week, these exercises are supervised by a physical or occupational therapist to stimulate the legs or arms respectively. Continued use of this combination of focal brain TMS therapy for somatosensory cortex stimulation together with rehabilitative and skill therapies have a desired outcome. The child learns to walk and use his hands to manipulate small objects and perform self-feeding. After one year of this intensive program, the child gains the ability to walk independently and to feed himself.

### Example 6

### TMS-Related Equipment

In a preferred mode of administration, focal noninvasive neurostimulation in accordance with the invention is painless and is accompanied by little if any side effect movements. One embodiment of the invention comprises an apparatus for providing noninvasive focal simulation in a reproducible manner; the apparatus is customized to the anatomy of the subject that is to receive the neurostimulation.

For example, the apparatus can comprise a cap, headgear or helmet (for convenience the term “cap” will be used hereinafter) that contains means for providing noninvasive focal stimulation of brain tissue; in one embodiment, these are TMS coils. The relatively small head sizes of pediatric subjects allows that smaller and lighter coils can be used to advantage; it es a further advantage of the smaller lighter coild that they fit more redilty in the cap of the invention. Presently available TMS coils can be used in the present invention. A preferred embodiment of a TMS coil is a “figure 8” design; the smallest area that can be stimulated with a figure 8 coil is within the current level of resolution and consistent with the area you need to stimulate in the sensory cortex. In a preferred embodiment, the cap also comprises a means to facilitate cooling of the neurostimulation means. In certain embodiments gas or liquid fluids are used to offset, insulate and/or dissipate heat induced by neurostimulation. Thermal insulative or thermal conductive materials are, as appreciated by those of skill in the art, used to advantage in caps of the invention in order to make the caps as comfortable as possible for the subject user.

There will be a power source that will energize the means for providing noninvasive focal stimulation of brain tissue. The power source may be AC or DC electrical current, be provided to the cap by physical connection such as from a battery, console or wall outlet. The cap may comprise a power source contained in the cap itself or the cap may receive energy by transmission through the ambient area, without the need for a physical connection. The cap and/or its power source can have computer software or programs that allow it to be pre-programmed. The software or programs can address monitoring and safety parameters. Fiduciary marks can be integrated into the cap. The position of coil(s) can be registered electronically and thus facitate uniform placement on the subject. Postprocessing software can link the 2D or 3D DTI data with where to position the TMS coils on the scalp. During periods of growth, the cap is reconfigured to suit the current size of the subject's head and brain. The reconfiguration takes place every 6-12 months or as otherwise needed.

### Example 7

### Neurostimulation Equipment and Combinations Thereof

Concurrent or contemporaneous with the neurostimulation of the invention, it can also be beneficial to perform tasks to facilitate or induce the development of skills. These tasks can include physical movements of the subject's body. These movements can comprise occupational therapy, physical therapy, strength-building tasks, stamina-building tasks, coordination-building tasks, etc.

In one embodiment, the neurostimulation of the invention is provided together with (concurrent or contemporaneous) provision of skill-building equipment. Examples of equipment to facilitate such skill-building are computer programs and corresponding equipment and stations that facilitate development of coordination between visual information and the position and or use of appendages such as feet, legs, hands and arms, for this purpose the head is considered an appendage as well. Equipment in accordance with the invention can comprise: equipment that is designed to facilitate movement and exercise of muscles, joints or body parts such as, without limitation, treadmill equipment, stairclimbing equipment, rowing equipment, ski simulation equipment and the like; devices that provide audio or video information; and combinations thereof. The skill-building equipment can be motorized or partially motorized in order to provide resistance or assistance. Combinations of various kinds of skill-building equipment disclosed herein are within the scope of the invention, it being understood that the equipment serves to exercise one or more muscle groups.

In addition, the neurostimulation means may be comprised with equipment to facilitate compliance by the users. The compliance-related features may include things to distract or entertain. This may be devices that emit music or sounds, radio, television, internet, telephone, toys, games or the like.

In one embodiment of the invention a means for providing neurostimulation is integrated with equipment to facilitate skill-building. The integrated equipment of the invention can be along the lines of a workstation where the subject receives neurostimulation together with other equipment to facilitate the building of skills. Pediatric subjects are a preferred group to undergo methods in accordance with the invention. The skill-building equipment for children can be designed to be user friendly and fun, such as play equipment or video games.

### Example 7

### Kits

The invention also comprises kits. In one embodiment, the kit includes a device in accordance with the invention, such as a computer program or a cap comprising a means for providing neurostimulation. In some embodiments, the kit comprises an outer container or package. The kit can comprise a container can be made of plastic, paper, cardboard, plastic, glass, metal foil, or other materials suitable for holding or separating materials.

The kits can contain one or more articles of the invention (equipment, compositions, software, respective instructions etc.). Replacement parts or an amount of disposables can be included in the kit. In certain kit embodiments, a composition, device, equipment and/or article etc. of the invention is provided together with instructions for administering it to a subject.

Instructions may include information about the proper use and/or effects of the composition, device, and/or article etc. In one embodiment, the instructions will include at least one of the following: description of equipment/composition/software etc. of the invention, dosage/treatment parameters and administration protocols, precautions, warnings, indications, counter-indications, overdosage information, adverse reactions, animal pharmacology, clinical studies, and/or references. The instructions may be printed directly on a container (when present), or as a label applied to the container, on as shrink wrap, or as a separate sheet, pamphlet, card, or folder supplied in or with the container. Thus, the instructions may be a separate item in the kit, or be imprinted, embossed, molded or otherwise affixed to another item in the kit; instructions may be printed on an outer container and also included as an insert item in the kit.

From the foregoing description, it will be apparent that variations and modifications may be made to the invention described herein to adopt it to various usages and conditions. Such embodiments are also within the scope of the following claims. The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The above examples are provided to illustrate the invention but not to limit its scope. Other variants of the invention will be readily apparent to one of ordinary skill in the art and are encompassed by the appended claims. All references, including patents, publications, databases and computer programs mentioned in this specification are herein incorporated by reference to the same extent as if each independent patent and publication was specifically and individually indicated to be incorporated by reference. The above examples are provided to illustrate the invention but not to limit its scope. Other variants of the invention will be readily apparent to one of ordinary skill in the art and are encompassed by the appended claims.

What is claimed is:

1. A method for stimulating nerve tissue in a sensory area of the brain of a subject with cerebral palsy:
determining that an afferent tract to a brain cortex sensory area of the subject manifests pathology;
identifying a sensory area of the subject's brain that receives information from the pathologic afferent tract;
administering to the subject a modality predicated on the existence of the sensory area that receives deficient afferent information.

2. The method of claim 1 wherein the determining step comprises diffusion tensor imaging.

3. The method of claim 1 wherein the determining step comprises determining that the afferent tract provides inadequate sensory information.

4. The method of claim 1 wherein the determining step comprises determining that the afferent tract provides abnormal sensory information.

5. The method of claim 1, where the administering step comprises providing stimulation to the specific sensory area of the subject's brain that is missing normal afferent information.

6. A method for stimulating a sensory cortical area of a subject's brain:
determining that afferent nerves to a sensory cortical area of the subject's brain manifest pathology;
identifying an area of the subject's sensory cortex that corresponds to the afferent nerve pathway;
mapping the sensory cortical area to the surface of the subject's head;
placing a means for noninvasive focal stimulation of internal nerve tissue at the mapped area of the subject's head;
stimulating noninvasively and focally the area of the sensory cortex that corresponds to pathologic efferent nerves without concomitantly stimulating brain tissue in a generalized manner.

7. The method of claim 6 wherein the subject has cerebral palsy or periventricular leukomalacia.

8. The method of claim 6 wherein the determining step comprises determining that the afferent nerves manifest pathology with diffusion tensor imaging (DTI).

9. The method of claim 6 wherein the determining step comprises: determining that the afferent nerves manifest a pathology comprising a paucity of sensory fibers.

10. The method of claim 6 wherein the determining step comprises: determining that the afferent nerves manifest a pathology comprising impaired conduction by sensory fibers.

11. The method of claim 6 wherein
the placing step comprises placing transcranial magnetic stimulation coils; and,
the stimulating step comprises stimulating the area of the sensory cortex with transcranial magnetic stimulation.

12. A method for eliciting efferent stimulation of motor fiber nerve tracts in subjects with cerebral palsy:
identifying a sensory area of the subject's brain that has a deficit;
mapping the sensory area to the surface of the subject's head;
placing a means for noninvasive focal stimulation of internal nerve tissue at the mapped area of the subject's head;
stimulating noninvasively and focally the sensory area of the subject's brain that has a deficit without concomitantly stimulating brain tissue in a generalized manner, and
eliciting from the area sensory area efferent stimuli along motor fibers.

13. The method of claim 12 wherein the eliciting step further comprises minimizing atrophy of the motor fibers thereby.

14. The method of claim 12 wherein the identifying step comprises: determining that the sensory area manifests a deficit with diffusion tensor imaging (DTI).

15. The method of claim 12 wherein the determining step comprises: determining that the sensory area manifests a deficit comprising a paucity of afferent sensory fibers.

16. The method of claim 12 wherein the determining step comprises: determining that the sensory area manifests a deficit comprising impaired conduction of afferent sensory fibers.

17. An apparatus for use in neurostimulation of a human subject's head, the apparatus comprising:
a body portion configured to fit about the upper portion of the subject's head, whereby the eyes, nose, mouth and preferably the ears are uncovered;
at least one device that upon activation induces noninvasive focal neurostimulation of the subject's brain;
means for containing the device is a secure manner, the containing means attached to or formed within the body portion.

18. The apparatus of claim 17 wherein the body portion comprises a cap.

19. The apparatus of claim 18 wherein the cap is fabric.

20. The apparatus of claim 17 wherein the body portion comprises thermal insulating material.

21. The apparatus of claim 17 wherein the body portion comprises thermal conductive material.

22. The apparatus of claim 17 wherein the neurostimulation device comprises a transcranial magnetic stimulation coil.

23. The apparatus of claim 22 wherein the coil is a figure eight coil.

24. The apparatus of claim 17 wherein the containing means comprises a pocket within the body portion.

25. the apparatus of claim 17 wherein the containing means comprises a mechanism for removably attaching the coil to the body portion.

26. the apparatus of claim 17 wherein the mechanism for removably attaching the coil to the body portion is a snap, hook, or Velcro component.

27. The apparatus of claim 17 further comprising a means for cooling the neurostimulation device.

28. The apparatus of claim 17 further comprising skill-building equipment.

29. The apparatus of claim 28 wherein the skill-building equipment comprises at least one of a computer program; computer keyboard; monitor; or equipment that is designed to facilitate movement and exercise of muscles, joints or body parts

30. The apparatus of claim 17 further comprising a means for entertaining or distracting the subject.

31. The apparatus of claim 30 wherein the means for entertaining or distracting the subject comprises a television screen, gaming device, a device to emit sounds such as music or speech, a rack to hold reading material.

32. The apparatus of claim 17 further comprising a container for shipping or storing the apparatus and instructions for use of the apparatus.

33. The apparatus of claim 32 wherein the instructions for use of the apparatus are on the container, in the container, or on the apparatus itself.

34. A method for evaluating the brain of a subject with cerebral palsy:
imaging one or more afferent tracts to a brain cortex sensory area of the subject;
determining that an afferent tract to a brain cortex sensory area of the subject manifests pathology; and,
whereby the existence of the pathologic afferent tract indicates that the subject has a sensory deficit that contributes to the subject's symptoms.

35. The method of claim 34 wherein the determining step comprises diffusion tensor imaging.

36. The method of claim 34 wherein the determining step comprises determining that the afferent tract provides inadequate sensory information.

37. The method of claim 34 wherein the determining step comprises determining that the afferent tract provides abnormal sensory information.

38. A method for designing treatment of a subject with cerebral palsy, the method comprising performing the method of claim 34, and further a step of devising a therapy that alleviates symptoms caused by the pathologic afferent tract.

39. The method of claim 38 further comprising identifying a specific sensory cortex area of the subject's brain that receives information from the pathologic afferent tract; and, the devising step comprises tailoring the therapy to utilize the specific sensory cortex area of the subject's brain.

40. The method of claim 39, where the devised therapy provides stimulation to the specific sensory area of the subject's brain that is missing normal afferent information.
